SEQUENCE LISTING (3 sequences)

SEQ ID NO: 1 - protein, 33 aa, Homo sapiens
HADGSFSDEMNTILDNLAARDFINWLIQTKITD

SEQ ID NO: 2 - protein, 34 aa, Artificial Sequence
  Synthetic
HXXGXFXXXXXXXXXXXXARXFIXWLIXTRITXX

SEQ ID NO: 3 - protein, 34 aa, Artificial Sequence
  Synthetic
HXXGXFXXXXXXXXXXXXAXXFIXWLIXTXITXX

(19) United States
(12) Patent Application Publication
(10) Pub. No.: US 2009/0011987 A1
(43) Pub. Date: Jan. 8, 2009

(54) Use of Glp-2 for the Treatment of Ischemia-Reperfusion Injury

(75) Inventor: Uffe Bang Olsen, Vallensbaek (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/914,786

(22) PCT Filed: May 19, 2006
(86) PCT No.: PCT/EP2006/062457

§ 371 Date: Dec. 10, 2007

(30) Foreign Application Priority Data
May 19, 2005 (EP) 05104280.2

(51) Int. Cl. A61K 38/17; A61P 9/02

(52) U.S. Cl. 514/12

(57) ABSTRACT

The present invention relates, inter alia, to the use of GLP-2 compounds in the treatment of ischemia-reperfusion injury in a subject.

### FIELD OF THE INVENTION

The present invention relates to a novel use of GLP-2 compounds in the treatment and prevention of conditions related to ischemia-reperfusion injuries.

### BACKGROUND OF THE INVENTION

Ischemia-reperfusion injury frequently occurs when the flow of blood to a region of the body is temporarily halted (ischemia) and then re-established (reperfusion). Ischemia-reperfusion injury can occur during certain surgical procedures, such as repair of certain aortic aneurysms and organ transplantation. Clinically ischemia-reperfusion injury in manifested by such complications as pulmonary dysfunction, including adult respiratory distress syndrome, renal dysfunction, consumptive coagulopathies including thrombocytopenia, fibrin deposition into the microvasculature and disseminated intravascular coagulopathy, transient and permanent spinal cord injury, cardiac arrhythmias and acute ischemic events, hepatic dysfunction including acute hepatocellular damage and necrosis, gastrointestinal dysfunction including hemorrhage and/or infarction, multisystem organ dysfunction (MSOD), such as Multiple Organ Failure (MOF), or acute systemic inflammatory distress syndromes (SIRS). The injury may occur in the parts of the body to which the blood supply was interrupted, or it can occur in parts fully supplied with blood during the period of ischemia.

International Patent Applications WO 97/31943, WO 98/08872, WO 96/32414, WO 98/03547, WO 99/43361, WO 97/39031, WO 04/035624, and WO 04/085471 disclose GLP-2 peptides including GLP-2 analogs and derivatives and make mention of the use of such peptides in the treatment of gastrointestinal disorders. International Patent Applications WO 99/43361 and WO 01/49314 disclose pharmaceutical compositions comprising GLP-2 peptides that also may be useful for such purposes.

There remains a need for methods of treatment and prevention of conditions related to ischemia-reperfusion injuries. The invention described herein provides such methods. These and other advantages of the invention, as well as additional inventive aspects and features, will be apparent from the description of the invention provided herein.

### SUMMARY OF THE INVENTION

The present invention relates, in a broad aspect, to the prevention and treatment of conditions related to ischemia-reperfusion injuries.

In a first exemplary aspect, the invention relates to the use of a GLP-2 compound for the preparation of a medicament for the treatment of ischemia-reperfusion injury in a subject. In one embodiment the subject is a human.

The GLP-2 compound composition typically and desirably also comprises one or more pharmaceutically acceptable vehicles, diluents, excipients, carriers, protectants, flavorants, preservatives, stabilizers, activity enhancers, buffers, colorants, wetting agents, lubricants, tabletting agents, solvents, solutes, anti-oxidants, biostatic agents, suspending agents, isotonic agents, thickening agents, adjuvants, emulsifiers, salts, aromatic agents, solubilizers, or any combination thereof. In these and other specific exemplary aspects, the GLP-2 compound in the composition is in a concentration of about 0.001-0.5 mg/ml.

In another exemplary aspect, the invention provides a method for the treatment of ischemia-reperfusion injury in a subject comprising administering to said subject a composition comprising a therapeutically or prophylactically effective amount of a GLP-2 compound, whereby at least one symptom of ischemia-reperfusion injury is alleviated.

In another exemplary aspect, the invention relates to a method for the treatment of ischemia-reperfusion injury in a subject comprising administering to said subject a composition comprising a therapeutically or prophylactically effective amount of a GLP-2 compound, whereby at least one symptom of ischemia-reperfusion injury is alleviated.

In another exemplary aspect, the invention relates to a method for protecting, preventing or reducing ischemia-reperfusion injury in a subject about to undergo a procedure capable of causing ischemia-reperfusion injury or in a subject who has already undergone such procedure in which ischemia-reperfusion injury has not yet occurred comprising administering to said subject an amount of a GLP-2 compound effective to prevent or reduce at least one symptom of ischemia-reperfusion injury.

### DETAILED DESCRIPTION OF THE INVENTION

The present invention comprises a method for treating ischemia-reperfusion injury comprising administering to a patient in need of such treatment an effective amount of a GLP-2 compound. Another aspect of this invention comprises a method for preventing ischemia-reperfusion injury in a patient about to undergo a procedure capable of causing ischemia-reperfusion injury or to a patient who has already undergone such procedure in which ischemia-reperfusion injury has not yet occurred comprising administering to the patient an effective amount of a GLP-2 compound.

One applications of this invention are preventing ischemia-reperfusion injury by administering the GLP-2 compound in conjunction with surgical repair of e.g. the thoracic or suprarenal aorta due to aneurysmal disease, but also in conjunction with those surgical procedures that induce or require transient occlusion or bypass of the visceral blood supply via the hepatic, renal and/or enteric arteries secondary to major organ transplant, including liver, kidney, small intestine, and pancreas as well as surgical procedures that result in the transient reduction or prevention of blood flow to the viscera including hepatic and biliary surgical resections, total or partial pancreatectomy (Whipple procedure), total and partial gastrectomy, esophagectomy, colorectal surgery, vascular surgery for mesenteric vascular disease, or abdominal insufflation during laparoscopic surgical procedures. Additional applications include trauma, such as blunt or penetrating trauma that results in interruption of blood flow to the viceral organs including those arising from penetrating wounds to the abdomen resulting from gun shot wounds, stab wounds or from penetrating wounds or blunt abdominal trauma secondary to deacceleration injury and/or motor vehicle accidents. Other applications include diseases or procedures that result in systemic hypotension that either disrupts or decreases the flow of blood to the visceral organs, including hemorrhagic shock due to blood loss, cardiogenic shock due to myocardial infarction or cardiac failure, neurogenic shock or anaphylaxis.

In one embodiment the ischemia-reperfusion injury is associated with a condition or disease selected from the group consisting of transplantation, inflammation, stroke, seizure, rheumatoid arthritis, atherosclerosis, cancer, dementia, diabetes, hypertensive crisis, ulcers, lupus, sickle cell anemia, ischemic bowel syndrome, pulmonary emboli, Ball's syndrome, pancreatitis, heart attack, and aging.

In one embodiment the ischemia-reperfusion injury is associated with a condition or disease selected from the group consisting of pulmonary dysfunction, including adult respiratory distress syndrome, renal dysfunction, consumptive coagulopathies including thrombocytopenia, fibrin deposition into the microvasculature and disseminated intravascular coagulopathy, transient and permanent spinal cord injury, cardiac arrhythmias and acute ischemic events, hepatic dysfunction including acute hepatocellular damage and necrosis, gastrointestinal dysfunction including hemorrhage and/or infarction, multisystem organ dysfunction (MSOD), such as Multiple Organ Failure (MOF) or acute systemic inflammatory distress syndromes (SIRS). The injury may occur in the parts of the body to which the blood supply was interrupted, or it can occur in parts fully supplied with blood during the period of ischemia.

The amount of a GLP-2 compound to be administered is preferably between 0.1 to 500 μg/kg of body weight, more preferably 1 to 50 μg/kg. Administration preferably takes place by intravenous, intramuscular or subcutaneous injection.

In those surgical procedures in which temporary or sustained disruption of blood flow is anticipated to occur, as before surgical repair of thoracoabdominal or supraceliac aneursymal disease, or surgical procedures to the abdomen that will necessarily include the transient reduction in visceral blood flow, or for organ transplantation, the GLP-2 compound is preferably given either as a single bolus injection one to zero hours before the ischemic event or as a continuous intravenous injection beginning one to zero hours before the ischemic event and extending during the perioperative period and continuing for at least eight hours after restoration of visceral blood flow. For individuals in whom disrupted visceral blood flow has already occurred, as in those individuals with trauma or injury to the visceral organs or their blood supply, or in patients with systemic hypotension due to shock, the GLP-2 compound would be preferably given either as a single bolus injection prior to or simultaneously with restoration of normal visceral blood flow or as a continuous intravenous injection prior to or simultaneously with restoration of normal visceral blood flow and extending for at least eight hours after restoration of visceral blood flow.

One aspect of the present invention relates to the use of a GLP-2 compound for the preparation of a medicament for the treatment of ischemia-reperfusion injury in a subject

In one embodiment of the invention, the medicament is administered intravenously, intramuscularly or subcutaneously.

In a further embodiment of the invention, the ischemic reperfusion injury is caused by or is expected to be caused by a major organ transplant, repair of an aneurysm, surgical repair of a thoracic aortic aneurysm, coronary artery bypass, a suprarenal aortic aneurysm, myocardial infarction, angina such as stable or unstable angina, such coronary artery spasm or unstable angina caused by coronary artery disease due to atherosclerosis, liver, kidney, small intestine, or pancreas transplant, hepatic and biliary surgical resections, total or partial pancreatectomy, total and partial gastrectomy, esophagectomy, colorectal surgery, vascular surgery for mesenteric vascular disease, mesenteric thrombus, mesenteric venous occlusion, abdominal insufflation during laparoscopic surgical procedures, blunt or penetrating trauma to the abdomen including gun shot wounds, stab wounds or penetrating wounds or blunt abdominal trauma secondary to deacceleration injury or motor vehicle accidents, hemorrhagic shock due to blood loss, cardiogenic shock due to myocardial infarction or cardiac failure, neurogenic shock or anaphylaxis, surgical treatment of arterial occlusion of limbs, cerebral infarction and intestinal infarction.

In a further embodiment of the invention, the medicament is administered prior to onset of ischemia.

In a further embodiment of the invention, the medicament is administered substantially concurrently with onset of ischemia.

In a further embodiment of the invention, the medicament is administered about 24 hours prior to onset of ischemia.

In a further embodiment of the invention, the formulation further includes a compound having an anti-ischemic effect.

In a further embodiment of the invention, the ischemia-reperfusion injury is in an organ selected from the group consisting of intestine, brain, heart, liver, kidney, cornea, lung and combinations thereof.

In a further embodiment of the invention, the ischemia-reperfusion injury is in the Intestine.

In a further embodiment of the invention, the ischemia-reperfusion injury is in an organ selected from the group consisting of brain, heart, liver, kidney, cornea, lung and combinations thereof.

In a further embodiment of the invention, the ischemia-reperfusion injury is in the brain.

In a further embodiment of the invention, the ischemia-reperfusion injury is in the heart.

In a further embodiment of the invention, the ischemia-reperfusion injury is the liver.

In a further embodiment of the invention, the ischemia-reperfusion injury is the kidney.

In a further embodiment of the invention, the ischemia-reperfusion injury is the cornea.

In a further embodiment of the invention, the ischemia-reperfusion injury is the lung.

As used herein, the term “GLP-2 compound” means any GLP-2 receptor agonist. The term encompasses GLP-2 peptides, and analogues thereof, as well as GLP-2 derivatives. A GLP-2 receptor agonist typically binds to a GLP-2 receptor, preferably with an affinity constant (K_{D}) or a potency (EC₅₀) of below about 1 μM, e.g. about 1 nM-750 nM, such as about 500 nM or less, about 250 nM or less, about 100 nM or less, about 75 nM or less, or about 50 nM or less. Examples of suitable GLP-2 compounds that can be useful in practicing many of the methods of the invention are disclosed in e.g. WO 96/29342, WO 97/31943, WO 98/08872, WO 98/03547, WO 96/32414, WO 97/39031, WO 04/035624, and WO 04/085471, the content of which are incorporated herein by reference in their entirety. In certain aspects, the GLP-2 compound is an intestinotrophic molecule, such as an intestinotrophic GLP-2 peptide.

The term “GLP-2 peptide” as used herein means any biologically active protein, polypeptide, or peptide (which terms are generally used interchangeably throughout unless otherwise stated or clearly contradicted by context) comprising the amino acid sequence 1-33 of native human GLP-2 (SEQ ID NO:1) or a sequence having at least about 65%, at least about 75%, at least about 80%, at least about 85%, at least about 90%, at least about 95%, or more (e.g., about 70-99%) amino acid sequence identity to this sequence. Thus, “GLP-2 peptides” include, but is not limited to, native human GLP-2 and analogs thereof. A GLP-2 peptide can have any suitable amino acid sequence or other features, so long as the peptide at least substantially retains (e.g., retains at least about 50%, at least about 75%, at least about 90%, about 100% or more, about 125% or more, about 150% or more, about 200% or more, etc.) of the ability to induce, promote, and/or enhance at least one physiological response associated with the administration of GLP-2 (e.g., the ability to detectably promote the growth of gastrointestinal tissue in a mammalian host over a period of time, the ability to detectably inhibit apoptosis of certain cells (as compared to the individual or a population of substantially similar individuals), the ability to detectably reduce intestinal permeability, the ability to detectably stimulate enterocyte glucose transport, the ability to detectably increase adenylate cyclase activity, and/or the ability to detectably inhibit gastric emptying and gastric acid secretion).

Human GLP-2 is a 33 amino acid residue peptide produced in intestinal L-cells and released following nutrient intake. The amino acid sequence of human GLP-2 is set forth in FIG. 1.

The term “GLP-2” as used herein refers to a protein comprising amino acids 1-33 of native human GLP-2 (SEQ ID NO:1) and proteins with a slightly modified amino acid sequence, for instance, proteins comprising a modified N-terminal end including N-terminal amino acid deletions or additions (so long as those proteins substantially retain the activity of GLP-2). “GLP-2” within the above definition also includes natural allelic variations that may exist and occur from one individual to another. Also, degree and location of glycosylation or other post-translation modifications may vary depending on the chosen host cells and the nature of the host cellular environment in which the GLP-2 is produced.

The terms “analog” or “analogs”, as used herein, designate a GLP-2 peptide having the sequence similar to SEQ ID NO:1, but wherein one or more amino acids of SEQ ID NO:1 (“the parent sequence”) have been substituted, deleted, and/or inserted and/or where one or more amino acid residues have been added to the N and/or C terminus of SEQ ID NO:1. The “analog” or “analogs” within this definition still have GLP-2 activity, desirably as measured by the ability to exert a trophic effect on the small or large intestine (although other suitable markers of GLP-2 activity also or alternatively can be used in other aspects). In one aspect an analog is about 70% identical with the sequence of SEQ ID NO:1. In another aspect an analog is about 80% identical with the sequence of SEQ ID NO:1. In yet another aspect an analog is about 90% identical with the sequence of SEQ ID NO:1. In a further aspect an analog is 95% identical with the sequence of SEQ ID NO:1.

In another aspect an analog is a GLP-2 peptide, wherein a total of up to ten amino acid residues of SEQ ID NO:1 have been exchanged with any amino acid residue. In a further aspect an analog is a GLP-2 peptide, wherein a total of up to five amino acid residues of SEQ ID NO:1 have been exchanged with any amino acid residue. In a further aspect an analog is a GLP-2 peptide, wherein a total of up to three amino acid residues of SEQ ID NO:1 have been exchanged with any amino acid residue. In a further aspect an analog is a GLP-2 peptide, wherein a total of up to two amino acid residues of SEQ ID NO:1 have been exchanged with any amino acid residue. In a further aspect an analog is a GLP-2 peptide, wherein a total of one amino acid residue of SEQ ID NO:1 have been exchanged with any amino acid residue. Thus, for example, in one aspect of the invention the GLP-2 compound is a GLP-2 peptide, wherein a total of up to 5 amino acid residues have been exchanged with any α-amino acid residue, such as 4 amino acid residues, 3 amino acid residues, 2 amino acid residues, or 1 amino acid residue.

In one aspects, the GLP-2 peptide used in the methods of the invention and/or included in the formulation of the invention, is a GLP-2 peptide with a half-life lower than about 2 hours. The “half-life” as used herein means the half-life of detectable functional GLP-2 compound in adult human plasma. A GLP-2 peptide with a half-life lower than about 2 hours according to the above definition includes wild type human GLP-2(1-33). In one embodiment the GLP-2 compound has a half-life lower than about 1 hour, such as lower than about 30 min, such as lower than about 20 min, such as lower than 10 min.

In particular aspects, the GLP-2 peptide used in the methods of the invention and/or included in the formulation of the invention comprises, consists, or consists essentially of an amino acid sequence according to the formula:

His-X²-X³-Gly-X⁵-Phe-X⁷-X⁸-X⁹-X¹⁰-X¹¹-X¹²-X¹³-X¹⁴-X¹⁵-X¹⁶-X¹⁷-X¹⁸-Ala-X²⁰-X²¹-Phe-Ile-X²⁴-Trp-Leu-Ile-X²⁸-Thr-Arg-Ile-Thr-X³³-X³⁴ (formula I)

or a fragment thereof, wherein “X” represents any suitable amino acid residue. In more particular aspects, X² is Ala, Val or Gly; X³ is Asp, or Glu; X⁵ is Ser, or Lys; X⁷ is Ser, or Lys; X⁸ is Asp, Glu, or Lys; X⁹ is Asp, Glu, or Lys; X¹⁰ is Met, Lys, Leu, Ile, or Nor-Leucine; X¹¹ is Asn, or Lys; X¹² is Thr, or Lys; X¹³ is Ile, or Lys; X¹⁴ is Leu, or Lys; X¹⁵ is Asp, or Lys; X¹⁶ is Asn, or Lys; X¹⁷ is Leu, or Lys; X¹⁸ is Ala, or Lys; X²⁰ is Arg, or Lys; X²¹ is Asp, or Lys; X²⁴ is Asn, or Lys; X²⁸ is Gln, or Lys; and/or X³³ is Asp, Glu, or Lys, X³⁴ is OH, Lys, Arg, Arg-Lys, Lys-Arg, Arg-Arg, or Lys-Lys.

In other particular aspects, the GLP-2 peptide used in the methods and/or included in the formulations of the invention comprises (includes), consists, or consists essentially of an amino acid sequence according to the formula:

His-X²-X³-Gly-X⁵-Phe-X⁷-X⁸-X⁹-X¹⁰-X¹¹-X¹²-X¹³-X¹⁴-X¹⁵-X¹⁶-X¹⁷-X¹⁸-Ala-X²⁰-X²¹-Phe-Ile-X²⁴-Trp-Leu-Ile-X²⁸-Thr-X³⁰-Ile-Thr-X³³-X³⁴ (formula II)

or a fragment thereof; wherein “X” represents any suitable amino acid residue. In particular aspects, X² is Ala, Val or Gly; X³ is Asp, or Glu; X⁵ is Ser, or Lys; X⁷ is Ser, or Lys; X⁸ is Asp, Glu, or Lys; X⁹ is Asp, Glu, or Lys; X¹⁰ is Met, Lys, Leu, Ile, or Nor-Leucine; X¹¹ is Asn, or Lys; X¹² is Thr, or Lys; X¹³ is Ile, or Lys; X¹⁴ is Leu, or Lys; X¹⁵ is Asp, or Lys; X¹⁶ is Asn, or Lys; X¹⁷ is Leu, or Lys; X¹⁸ is Ala, or Lys; X²⁰ is Arg, or Lys; X²¹ is Asp, or Lys; X²⁴ is Asn, or Lys; X²⁸ is Gln, or Lys; X³⁰ is Arg, or Lys; X³³ is Asp, Glu, or Lys, and/or X³⁴ is OH, Lys, Arg, Arg-Lys, Lys-Arg, Arg-Arg, or Lys-Lys.

With reference to either formula I or formula II, GLP-2 peptides useful in specific method and/or formulation of aspects of the invention can be characterized as follows. In one aspect X² is Ala. In one aspect X² is Gly. In one aspect X³ is Asp. In one aspect X³ is Glu. In one aspect X⁵ is Ser. In one aspect X⁷ is Ser. In one aspect X⁸ is Asp. In one aspect X⁸ is Glu. In one aspect X⁹ is Asp. In one aspect X⁹ is Glu. In one aspect X¹⁰ is selected from the group consisting of Met, Leu, Ile, and Nor-Leucine. In one aspect X¹¹ is Asn. In one aspect X¹² is Thr. In one aspect X¹³ is Ile. In one aspect X¹⁴ is Leu. In one aspect X¹⁵ is Asp. In one aspect X¹⁶ is Asn. In one aspect X¹⁷ is Leu. In one aspect X¹⁸ is Ala. In one aspect X²¹ is Asp. In one aspect X²⁴ is Asn. In one aspect X²⁸ is Gln. In one aspect X³³ is Asp. In one aspect X³³ is Glu. GLP-2 peptides suitable for use in methods and/or formulations of the invention can be characterized by any suitable combination of such specific attributes.

In certain aspects, GLP-2 peptides used in the methods and/or formulations of the invention (and comprising, consisting, or consisting essentially of an amino acid sequence according to either formula I or formula II) can be characterized on the basis that at least one amino acid independently selected from the list consisting of X⁵, X⁷, X⁸, X⁹, X¹⁰, X¹¹, X¹², X¹³, X¹⁴, X¹⁵, X¹⁶, X¹⁷, X¹⁸, X²⁰, X²¹, X²⁴, X²⁸, and X³³ is a Lys. In one aspect the amino acid independently selected from the list consisting of X⁵, X⁷, X⁸, X⁹, X¹⁰, X¹¹, X¹², X¹³, X¹⁴, X¹⁵, X¹⁶, X¹⁷, X¹⁸, X²⁰, X²¹, X²⁴, X²⁸, and X³³ is Lys. In one aspect the amino acid X⁵ is Lys. In one aspect the amino acid X⁷ is Lys. In one aspect the amino acid X⁸ is Lys. In one aspect the amino acid X⁹ is Lys. In one aspect the amino acid X¹⁰ is Lys. In one aspect the amino acid X¹¹ is Lys. In one aspect the amino acid X¹² is Lys. In one aspect the amino acid X¹³ is Lys. In one aspect the amino acid X¹⁴ is Lys. In one aspect the amino acid X¹⁵ is Lys. In one aspect the amino acid X¹⁶ is Lys. In one aspect the amino acid X¹⁷ is Lys. In one aspect the amino acid X¹⁸ is Lys. In one aspect the amino acid X²⁰ is Lys. In one aspect the amino acid X²¹ is Lys. In one aspect the amino acid X²⁴ is Lys. In one aspect the amino acid X²⁸ is Lys. In one aspect the amino acid X³⁰ is Lys. In one aspect the amino acid X³⁰ is Arg. In one aspect the amino acid X³³ is Lys. In one aspect the amino acid X³³ is Lys. In one aspect the amino acid X³⁴ is Lys. In one aspect the amino acid X³⁴ is Arg. As with other characterizations of the variable amino acid residues in formula I and formula II provided herein, such GLP-2 peptides also or alternatively can be characterized by any suitable combination of these characteristics.

The phrase “a fragment thereof”, as used herein, means any fragment (i.e., portion) of the referenced peptide or amino acid sequence (e.g., a fragment can refer to a portion of a formula I or formula II sequence) that is at least about 15 amino acids in length and exhibits (or at least substantially retains) some GLP-2 biological activity in a mammalian host. GLP-2 activity may be measured be GLP-2 receptor binding affinity or by any of the other measures of GLP-2 activity described herein (e.g., the detectable promotion of tissue growth in the small intestine). In one aspect the fragment comprises at least about 20 amino acids of the “parent” peptide or sequence (e.g., about 20-30 amino acids of native GLP-2). In another aspect, the fragment comprises at least about 25 amino acids of the parent. In one aspect the fragment comprises at least about 30 amino acids of the parent. The term “fragment” is used for convenience and is not meant to impart or imply any limitation on how such peptides are made or limit any other characteristics of such peptides (thus, for example, a peptide comprising a “fragment” of formula I, may actually comprise an amino acid sequence larger than formula I, for example in the context of a fusion protein comprising the “fragment” sequence).

In one aspect, the invention provides methods of using peptides comprising or consisting of a fragment and formulations that relate to peptides comprising or consisting of a fragment, wherein the fragment corresponds to formula I or formula II except for one amino acid deletion in the C-terminus, two amino acid deletions in the C-terminus, three amino acid deletions in the C-terminus, or four amino acid deletions in the C-terminus. In another aspect the fragment also or alternatively varies from formula I or formula II by one amino acid deletion in the N-terminus, two amino acid deletions in the N-terminus, three amino acid deletions in the N-terminus, or four amino acid deletions in the N-terminus.

The term “derivative” is used in the present text to designate a peptide in which one or more of the amino acid residues have been chemically modified, e.g. by alkylation, acylation, ester formation, or amide formation. The term “a GLP-2 derivative” is used in the present text to designate a derivative of a GLP-2 peptide. In one aspect the GLP-2 derivative according to the present invention has GLP-2 activity as measured by, for example, the ability to bind a GLP-2 receptor (GLP-2R) and/or exert trophic effects on the small intestine or large intestine. In one aspect the GLP-2 receptor used to measure GLP-2 activity is selected from the list consisting of rat GLP-2R, mouse GLP-2R, and human GLP-2R.

The term “lipophilic substituent” refers to a lipophilic group or moiety comprising about 4-40 carbon atoms and having a solubility in water at about 20° C. in the range from about 0.1 mg/100 ml water to about 250 mg/100 ml water, such as in the range from about 0.3 mg/100 ml water to about 75 mg/100 ml water. For instance, octanoic acid (C8) has a solubility in water at 20° C. of 68 mg/100 ml, decanoic acid (C10) has a solubility in water at 20° C. of 15 mg/100 ml, and octadecanoic acid (C18) has a solubility in water at 20° C. of 0.3 mg/100 ml. A simple system is used in the following to describe peptides, fragments, analogs, and derivatives of GLP-2 herein which will be readily apparent to the ordinarily skilled artisan. By way of illustration, R20K-GLP-2(1-31) designates a fragment of GLP-2 formally wherein the amino acid residues at positions 32 and 33 of SEQ ID NO:1 are deleted (“1-31”) and the naturally occurring amino acid residue arginine at position 20 of SEQ ID NO:1 is substituted by a lysine (“R20K”). Similarly, R20K(N^{ε}-tetradecanoyl)/K30R-GLP-2(1-33) designates a derivative of a GLP-2 peptide analog formally derived from GLP-2 by exchange of the naturally occurring amino acid residue lysine in position 30 of SEQ ID NO:1 with an arginine residue and exchange of the naturally occurring amino acid residue arginine in position 20 of SEQ ID NO:1 with a lysine residue and tetradecanoylation of the ε-amino group of the lysine residue in position 20 relative to the amino acid sequence of SEQ ID NO:1. Similarly, L17K(3-(hexadecanoylamino)propionyl)/K30R-GLP-2(1-33) designates a derivative of a GLP-2 peptide analog formally derived from GLP-2 by exchange of the naturally occurring amino acid residue lysine in position 30 of SEQ ID NO:1 with an arginine residue and exchange of the naturally occurring amino acid residue leucine in position 17 of SEQ ID NO:1 with a lysine residue and hexadecanoylation of the ε-amino group of the lysine residue in position 17 relative to the amino acid sequence of SEQ ID NO:1 by means of the spacer β-alanine (see, e.g., FIGS. 5 and 6).

The term “treatment” is meant to include both the prevention and minimization of the referenced disease, disorder, or condition (i.e., “treatment” refers to both prophylactic and therapeutic administration of a GLP-2 compound/composition unless otherwise indicated or clearly contradicted by context; however, therapeutic administration of GLP-2 compounds/compositions and prophylactic administration of GLP-2 compounds/compositions can separately be considered unique aspects of the invention). For example, in one aspect the invention provides a method of treating a condition resulting in the prevention of an expected ischemia-reperfusion injury that, without treatment, could lead to intestinal failure.

To obtain a protracted profile of action, a lipophilic substituent can be attached to the GLP-2 peptide to obtain a lipophilic GLP-2 derivative (such a lipophilic substituent can, for example, comprise about 4-40 carbon atoms, such as about 8-25 carbon atoms or about 10-20 carbon atoms, although other suitable sizes also can be used). In one aspect of the invention the lipophilic substituent comprises about 8 to about 40 carbon atoms; in another aspect about 10 to about 24 carbon atoms; in yet another aspect about 12 to about 24 carbon atoms; in yet another aspect 12 to 20 carbon atoms; in still another aspect about 12 to about 18 carbon atoms; and in yet a further aspect about 14 to about 18 carbon atoms. The size and character of a specific lipophilic substituent can impart advantageous properties to the GLP-2 peptide and accordingly can be considered a unique feature of such peptides.

The lipophilic substituent may be attached to any suitable amino group of the GLP-2 moiety or sequence by any suitable means, typically by way of a carboxyl group of the lipophilic substituent which forms an amide bond with an amino group of the amino acid residue to which it is attached. As an alternative, the lipophilic substituent may be attached to an amino acid in such a way that an amino group of the lipophilic substituent forms an amide bond with a carboxyl group of the amino acid. As a further option, the lipophillic substituent may be linked to the GLP-2 moiety via an ester bond. Formally, the ester can be formed either by reaction between a carboxyl group of the GLP-2 moiety and a hydroxyl group of the substituent-to-be or by reaction between a hydroxyl group of the GLP-2 moiety and a carboxyl group of the substituent-to-be. As a further alternative, the lipophilic substituent can be an alkyl group which is introduced into a primary amino group of the GLP-2 moiety. In certain aspects of the invention a lipophilic substituent is attached to a Lys residue of the GLP-2 sequence.

In certain aspects, the GLP-2 compound is at least substantially isolated and in others the GLP-2 compound can be considered “isolated.” The term “isolated GLP-2 compound” refers to a GLP-2 compound that is at least about 50%, typically at least about 65%, and more typically at least about 75% or more free of undesirable molecules (typically such undesirable molecules are biomolecules such as nonassociated polynucleotides, lipids, carbohydrates or other materials (i.e., contaminants) that the GLP-2 compound is naturally associated with). Purity can be measured by any suitable technique including, e.g., electrophoretic analysis, antibody-based analytical techniques, mass spectrometry, differential (e.g., affinity or mass based) centrifugation, and the like. In one aspect the GLP-2 compound is an isolated GLP-2 peptide; in another aspect the GLP-2 compound is an isolated GLP-2 derivative; in yet other aspects the isolated GLP-2 compound is an isolated nonpeptide GLP-2 compound (e.g., a “small molecule” GLP-2 receptor agonist, a nucleic acid encoding a GLP-2 antibody, GLP-2 receptor antibody, or other GLP-2 peptide agonist, or an antisense or interfering nucleic acid molecule (e.g., a GLP-2 expression modulating siRNA molecule). Preferably, the isolated compound or polypeptide is substantially free from any other contaminating polypeptides or other contaminants that are found in its natural environment which would interfere with its therapeutic, diagnostic, prophylactic or research use. Preferably, the isolated compound or polypeptide has been separated from at least about 80%, such as at least about 90%, at least about 95%, or more (desirably detectably free from all) free and undesired polynucleotides, lipids, carbohydrates or other materials (i.e., contaminants).

In specific aspects of the invention the GLP-2 compound is selected from a GLP-2 compound as disclosed in any of International Patent Applications WO 97/31943, WO 98/08872, WO 96/32414, WO 98/03547, WO 99/43361, and WO 97/39031.

In specific aspects of the invention the GLP-2 compound is selected from GLP-2(1-33), 34R-GLP-2(1-34), A2G-GLP-2(1-33), A2G/34R-GLP-2(1-34), K30R-GLP-2(1-33); S5K-GLP-2(1-33); S7K-GLP-2(1-33); D8K-GLP-2(1-33); E9K-GLP-2(1-33); M10K-GLP-2(1-33); N11K-GLP-2(1-33); T12K-GLP-2(1-33); I13K-GLP-2(1-33); L14K-GLP-2(1-33); D15K-GLP-2(1-33); N16K-GLP-2(1-33); L17K-GLP-2(1-33); A18K-GLP-2(1-33); D21K-GLP-2(1-33); N24K-GLP-2(1-33); Q28K-GLP-2(1-33); S5K/K30R-GLP-2(1-33); S7K/K30R-GLP-2(1-33); D8K/K30R-GLP-2(1-33); E9K/K30R-GLP-2(1-33); M10/K30R-GLP-2(1-33); N11K/K30R-GLP-2(1-33); T12K/K30R-GLP-2(1-33); I13K/K30R-GLP-2(1-33); L14K/K30R-GLP-2(1-33); D15K/K30R-GLP-2(1-33); N16K/K30R-GLP-2(1-33); L17K/K30R-GLP-2(1-33); A18K/K30R-GLP-2(1-33); D21K/K30R-GLP-2(1-33); N24K/K30R-GLP-2(1-33); Q28K/K30R-GLP-2(1-33); K30R/D33K-GLP-2(1-33); D3E/K30R/D33E-GLP-2(1-33); D3E/S5K/K30R/D33E-GLP-2(1-33); D3E/S7K/K30R/D33E-GLP-2(1-33); D3E/D8K/K30R/D33E-GLP-2(1-33); D3E/E9K/K30R/D33E-GLP-2(1-33); D3E/M10K/K30R/D33E-GLP-2(1-33); D3E/N11K/K30R/D33E-GLP-2(1-33); D3E/T12K/K30R/D33E-GLP-2(1-33); D3E/I13K/K30R/D33E-GLP-2(1-33); D3E/L14K/K30R/D33E-GLP-2(1-33); D3E/D15K/K30R/D33E-GLP-2(1-33); D3E/N16K/K30R/D33E-GLP-2(1-33); D3E/L17K/K30R/D33E-GLP-2(1-33); D3E/A18K/K30R/D33E-GLP-2(1-33); D3E/D21K/K30R/D33E-GLP-2(1-33); D3E/N24K/K30R/D33E-GLP-2(1-33); D3E/Q28K/K30R/D33E-GLP-2(1-33); and combinations of any thereof.

As mentioned elsewhere herein, the GLP-2 compound in certain aspects can advantageously be a GLP-2 derivative. Any suitable GLP-2 derivative can be used in the methods and/or act as an active ingredient in the formulations of the invention. In a particular aspect, the GLP-2 derivative comprises a GLP-2 peptide (i.e., comprises a GLP-2-like amino acid sequence) according to formula II or a fragment thereof. In specific aspects, the GLP-2 derivative comprises a formula II amino acid sequence or formula II amino acid sequence fragment characterized by one or more of the following: X² is Ala, Val or Gly; X³ is Asp, or Glu; X⁵ is Ser, or Lys; X⁷ is Ser, or Lys; X⁸ is Asp, Glu, or Lys; X⁹ is Asp, Glu, or Lys; X¹⁰ is Met, Lys, Leu, Ile, or Nor-Leucine; X¹¹ is Asn, or Lys; X¹² is Thr, or Lys; X¹³ is Ile, or Lys; X¹⁴ is Leu, or Lys; X¹⁵ is Asp, or Lys; X¹⁶ is Asn, or Lys; X¹⁷ is Leu, or Lys; X¹⁸ is Ala, or Lys; X²⁰ is Arg, or Lys; X²¹ is Asp, or Lys; X²⁴ is Asn, or Lys; X²⁸ is Gln, or Lys; X³⁰ is Arg, or Lys; X³³ is Asp, Glu, or Lys; and X³⁴ is OH, Lys, Arg, Arg-Lys, Lys-Arg, Arg-Arg, or Lys-Lys In one aspect of the invention the GLP-2 compound is a GLP-2 derivative comprising a GLP-2 peptide, wherein a lipophilic substituent is attached to one or more amino acid residues at a position relative to the amino acid sequence of SEQ ID NO:1 selected from the list consisting of S5, S7, D8, E9, M10, N11, T12, I13, L14, D15, N16, L17, A18, D21, N24, and Q28. In one aspect, a lipophilic substituent is attached to an amino acid residues at the position S5 relative to the amino acid sequence of SEQ ID NO:1. In another aspect, a lipophilic substituent is attached to an amino acid residues at the position S7 relative to the amino acid sequence of SEQ ID NO:1. In one aspect, a lipophilic substituent is attached to an amino acid residues at the position D8 relative to the amino acid sequence of SEQ ID NO:1. In one aspect a lipophilic substituent is attached to an amino acid residues at the position E9 relative to the amino acid sequence of SEQ ID NO:1. In one aspect a lipophilic substituent is attached to an amino acid residues at the position M10 relative to the amino acid sequence of SEQ ID NO:1. In one aspect a lipophilic substituent is attached to an amino acid residues at the position N11 relative to the amino acid sequence of SEQ ID NO:1. In one aspect a lipophilic substituent is attached to an amino acid residues at the position T12 relative to the amino acid sequence of SEQ ID NO:1. In one aspect a lipophilic substituent is attached to an amino acid residues at the position I13 relative to the amino acid sequence of SEQ ID NO:1. In one aspect a lipophilic substituent is attached to an amino acid residues at the position L14 relative to the amino acid sequence of SEQ ID NO:1. In one aspect a lipophilic substituent is attached to an amino acid residues at the position D15 relative to the amino acid sequence of SEQ ID NO:1. In one aspect a lipophilic substituent is attached to an amino acid residues at the position N16 relative to the amino acid sequence of SEQ ID NO:1. In one aspect a lipophilic substituent is attached to an amino acid residues at the position L17 relative to the amino acid sequence of SEQ ID NO:1. In one aspect a lipophilic substituent is attached to an amino acid residues at the position A18 relative to the amino acid sequence of SEQ ID NO:1. In one aspect a lipophilic substituent is attached to an amino acid residues at the position D21 relative to the amino acid sequence of SEQ ID NO:1. In one aspect a lipophilic substituent is attached to an amino acid residues at the position N24 relative to the amino acid sequence of SEQ ID NO:1. In one aspect a lipophilic substituent is attached to an amino acid residues at the position Q28 relative to the amino acid sequence of SEQ ID NO:1. It is to be understood that an amino acid residues at the position relative to the amino acid sequence of SEQ ID NO:1 may be any amino acid residue and not only the amino acid residue naturally present at that position. In one aspect the lipophilic substituent is attached to a lysine. GLP-2 derivatives can be characterized by any combination of these features.

In a further aspect, the lipophilic substituent may be attached to the GLP-2 peptide by means of a spacer in such a way that a carboxyl group of the spacer forms an amide bond with an amino group of the GLP-2 peptide. A spacer typically contains at least two functional groups, one to attach to a functional group of the lipophilic substituent and the other to a functional group of the parent GLP-2 peptide. The term “spacer” is used in the present text to designate a bivalent moiety which contain at least two functional groups, one to attach to a functional group of the lipophilic substituent and the other to a functional group of the GLP-2 compound.

Examples of suitable spacers include succinic acid, lysyl, glutamyl, asparagyl, glycyl, beta-alanyl and gamma-aminobutanoyl, and dipeptide spacers (such as Gly-Lys), each of which constitutes an individual aspect of the invention. Thus, a spacer can be one of these recited amino acid residues or another suitable amino acid. In a further aspect of the invention, the spacer is an amino acid residue (typically a naturally occurring amino acid residue) except Cys or Met. In another aspect, the spacer is a dipeptide such as Gly-Lys. In a further aspect the spacer is selected from lysyl, glutamyl, asparagyl, glycyl, beta-alanyl and gamma-aminobutanoyl, each of which constitutes an individual aspect. Typically used spacers are glutamyl, aminobutyroyl, and beta-alanyl (beta-Ala). When the spacer is succinic acid, one carboxyl group thereof may form an amide bond with an amino group of the amino acid residue, and the other carboxyl group thereof may form an amide bond with an amino group of the lipophilic substituent. When the spacer is lysyl, glutamyl, asparagyl, glycyl, beta-alanyl or gamma-aminobutanoyl, the carboxyl group thereof may form an amide bond with an amino group of the amino acid residue, and the amino group thereof may form an amide bond with a carboxyl group of the lipophilic substituent. When Lys is used as the spacer, a further spacer may in some instances be inserted between the ε-amino group of Lys and the lipophilic substituent. In one preferred aspect, such a further spacer is succinic acid which forms an amide bond with the ε-amino group of Lys and with an amino group present in the lipophilic substituent. In another preferred aspect such a further spacer is Glu or Asp which forms an amide bond with the ε-amino group of Lys and another amide bond with a carboxyl group present in the lipophilic substituent, that is, the lipophilic substituent is a N^{ε}-acylated lysine residue. In an aspect, the spacer is an amino acid residue except Cys or Met, or a dipeptide such as Gly-Lys. For purposes of the present invention, the phrase “a dipeptide such as Gly-Lys” means any combination of two amino acids except Cys or Met, typically a dipeptide wherein the C-terminal amino acid residue is Lys, His or Trp, typically Lys, and the N-terminal amino acid residue is Ala, Arg, Asp, Asn, Gly, Glu, Gln, Ile, Leu, Val, Phe, Pro, Ser, Tyr, Thr, Lys, His or Trp. Typically, an amino group of the GLP-2 compound forms an amide bond with a carboxylic group of the amino acid residue or dipeptide spacer, and an amino group of the amino acid residue or dipeptide spacer forms an amide bond with a carboxyl group of the lipophilic substituent.

In another aspect, the spacer is an unbranched alkane α,ω-dicarboxylic acid group comprising from about 1 to 7 methylene groups (e.g., 1-5 methylene groups, 2-4 methylene groups, 2 or 3 methylene groups, etc.), which spacer typically forms a bridge between an amino group of the parent peptide and an amino group of the lipophilic substituent. Typically, the spacer is succinic acid.

The lipophilic substituent(s) typically contain a functional group which can be attached to one of the following functional groups of an amino acid of the parent GLP-2 peptide: (a) the amino group attached to the alpha-carbon of the N-terminal amino acid, (b) the carboxy group attached to the alpha-carbon of the C-terminal amino acid, (c) the epsilon-amino group of any Lys residue, (d) the carboxy group of the R group of any Asp and Glu residue, (e) the hydroxy group of the R group of any Tyr, Ser and Thr residue, (f) the amino group of the R group of any Trp, Asn, Gln, Arg, and His residue, or (g) the thiol group of the R group of any Cys residue. In a further aspects of the invention, a lipophilic substituent is attached to the carboxy group of the R group of any Asp and Glu residue; a lipophilic substituent is attached to the carboxy group attached to the alpha-carbon of the C-terminal amino acid; or a lipophilic substituent is attached to the epsilon-amino group of any Lys residue.

In one aspect of the invention the spacer is an amino acid residue except a Cys residue, or a dipeptide. Examples of suitable spacers include (among others described elsewhere herein) β-alanine, gamma-aminobutyric acid (GABA), γ-glutamic acid, succinic acid, Lys, Glu or Asp, or a dipeptide such as Gly-Lys. When the spacer is Lys, Glu or Asp, the carboxyl group thereof may form an amide bond with an amino group of the amino acid residue, and the amino group thereof may form an amide bond with a carboxyl group of the lipophilic substituent. When Lys is used as the spacer, a further spacer may in some instances be inserted between the ε-amino group of Lys and the lipophilic substituent. In one aspect, such a further spacer is succinic acid which forms an amide bond with the ε-amino group of Lys and with an amino group present in the lipophilic substituent. In another aspect such a further spacer is Glu or Asp which forms an amide bond with the ε-amino group of Lys and another amide bond with a carboxyl group present in the lipophilic substituent, that is, the lipophilic substituent is a N^{ε}-acylated lysine residue.

In one aspect of the invention the spacer is selected from β-alanine, gamma-aminobutyric acid (GABA), γ-glutamic acid, Lys, Asp, Glu, a dipeptide containing Asp, a dipeptide containing Glu, or a dipeptide containing Lys. These and other specific spacers described herein can impart unique characteristics. As such, GLP-2 peptides comprising a β-alanine spacer can be considered a unique aspect of the invention apart from, for example, GLP-2 peptides comprising a gamma-aminobutyric acid (GABA) spacer and/or GLP-2 peptides comprising a γ-glutamic acid spacer.
Each lipophilic substituent typically contains a functional group which may be attached to a functional group of an amino acid of the parent GLP-2 peptide. For example, a lipophilic substituent may contain a carboxyl group which can be attached to an amino group of the parent GLP-2 peptide by means of an amide bond.

As discussed elsewhere herein, the spacer and GLP-2 peptide or GLP-2 amino acid sequence can be associated in any suitable manner. Thus, for example, in one aspect a carboxyl group of the parent GLP-2 peptide forms an amide bond with an amino group of a spacer, and the carboxyl group of the amino acid or dipeptide spacer forms an amide bond with an amino group of the lipophilic substituent, whereas, in another exemplary aspect, an amino group of the parent GLP-2 peptide forms an amide bond with a carboxylic group of a spacer, and an amino group of the spacer forms an amide bond with a carboxyl group of the lipophilic substituent.

In one aspect of the invention the lipophilic substituent comprises a partially or completely hydrogenated cyclopentanophenathrene skeleton. In another aspect of the invention the lipophilic substituent is an straight-chain or branched alkyl group.

In one aspect of the invention the lipophilic substituent is the acyl group of a straight-chain or branched fatty acid. In one aspect of the invention the acyl group of a lipophilic substituent is selected from the group comprising CH₃(CH₂)ₙCO—, wherein n is 4 to 38, such as CH₃(CH₂)₆CO—, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₁₈CO—, CH₃(CH₂)₂₀CO— and CH₃(CH₂)₂₂CO—.

In one aspect of the invention the lipophilic substituent is an acyl group of a straight-chain or branched alkane α,ω-dicarboxylic acid.

In one aspect of the invention the acyl group of the lipophilic substituent is selected from the group comprising HOOC(CH₂)ₘCO—, wherein m is 4 to 38, such as HOOC(CH₂)₁₄CO—, HOOC(CH₂)₁₆CO—, HOOC(CH₂)₁₈CO—, HOOC(CH₂)₂₀CO— and HOOC(CH₂)₂₂CO—.

In one aspect of the invention the lipophilic substituent is a group of the formula CH₃(CH₂)ₚ((CH₂)_{q}COOH)CHNH—CO(CH₂)₂CO—, wherein p and q are integers and p+q is an integer of from about 8 to about 40, such as from about 12 to about 35.

In one aspect of the invention the lipophlic substituent is a group of the formula CH₃(CH₂)ᵣCO—NHCH(COOH)(CH₂)₂CO—, wherein r is an integer of from about 10 to about 24.

In one aspect of the invention the lipophilic substituent is a group of the formula CH₃(CH₂)ₛCO—NHCH((CH₂)₂COOH)CO—, wherein s is an integer of from about 8 to about 24.

In one aspect of the invention the lipophilic substituent is a group of the formula COOH(CH₂)ₜCO— wherein t is an integer of from about 8 to about 24.

In one aspect of the invention the lipophilic substituent is a group of the formula —NHCH(COOH)(CH₂)₄NH—CO(CH₂)ᵤCH₃, wherein u is an integer of from about 8 to about 18.

In one aspect of the invention the lipophilic substituent is a group of the formula —NHCH(COOH)(CH₂)₄NH—COCH((CH₂)₂COOH)NH—CO(CH₂)_{w}CH₃, wherein w is an integer of from about 10 to about 16.

In one aspect of the invention the lipophilic substituent is a group of the formula —NHCH(COOH)(CH₂)₄NH—CO(CH₂)₂CH(COOH)NH—CO(CH₂)ₓCH₃, wherein x is an integer of from about 10 to about 16.

In one aspect of the invention the lipophilic substituent is a group of the formula —NHCH(COOH)(CH₂)₄NH—CO(CH₂)₂CH(COOH)NHCO(CH₂)_{y}CH₃, wherein y is zero or an integer of from about 1 to about 22.

In one aspect of the invention the lipophilic substituent is N-Lithocholoyl.

In one aspect of the invention the lipophilic substituent is N-Choloyl.

A GLP-2 derivative can comprise any suitable number of lipophilic substituents alone or in combination with other associated/conjugated groups/moieties. In one exemplary aspect of the invention the GLP-2 derivative has one lipophilic substituent. In another aspect of the invention the GLP-2 derivative has at least two (e.g., 2, 3, or 4) lipophilic substituents. In a further aspect of the invention the GLP-2 derivative has at least three lipophilic substituents. In yet another aspect of the invention the GLP-2 derivative has at least four lipophilic substituents (e.g., 4, 5, or more).

In one aspect of the invention the GLP-2 derivative is selected from:

S5K(3-(hexadecanoylamino)propionyl)-GLP-2(1-33);
S7K(3-(hexadecanoylamino)propionyl)-GLP-2(1-33);
D8K(3-(hexadecanoylamino)propionyl)-GLP-2(1-33);
E9K(3-(hexadecanoylamino)propionyl)-GLP-2(1-33);
M10K(3-(hexadecanoylamino)propionyl)-GLP-2(1-33);
N11K(3-(hexadecanoylamino)propionyl)-GLP-2(1-33);
T12K(3-(hexadecanoylamino)propionyl)-GLP-2(1-33);
I13K(3-(hexadecanoylamino)propionyl)-GLP-2(1-33);
L14K(3-(hexadecanoylamino)propionyl)-GLP-2(1-33);
D15K(3-(hexadecanoylamino)propionyl)-GLP-2(1-33);
N16K(3-(hexadecanoylamino)propionyl)-GLP-2(1-33);
L17K(3-(octanoylamino)propionyl)-GLP-2(1-33);
L17K(3-(nonanoylamino)propionyl)-GLP-2(1-33);
L17K(3-(decanoylamino)propionyl)-GLP-2(1-33);
L17K(3-(undecanoylamino)propionyl)-GLP-2(1-33);
L17K(3-(dodecanoylamino)propionyl)-GLP-2(1-33);
L17K(3-(tridecanoylamino)propionyl)-GLP-2(1-33);
L17K(3-(tetradecanoylamino)propionyl)-GLP-2(1-33);
L17K(3-(pentadecanoylamino)propionyl)-GLP-2(1-33);
L17K(3-(hexadecanoylamino)propionyl)-GLP-2(1-33);
L17K(3-(heptadecanoylamino)propionyl)-GLP-2(1-33);
L17K(3-(octadecanoylamino)propionyl)-GLP-2(1-33);
L17K(3-(nonadecanoylamino)propionyl)-GLP-2(1-33);
L17K(3-(eicosanoylamino)propionyl)-GLP-2(1-33);
L17K((S)-4-carboxy-4-(octanoylamino)butanoyl)-GLP-2(1-33);
L17K((S)-4-carboxy-4-(nonanoylamino)butanoyl)-GLP-2(1-33);
L17K((S)-4-carboxy-4-(decanoylamino)butanoyl)-GLP-2(1-33);
L17K((S)-4-carboxy-4-(undecanoylamino)butanoyl)-GLP-2(1-33);
L17K((S)-4-carboxy-4-(dodecanoylamino)butanoyl)-GLP-2(1-33);
L17K((S)-4-carboxy-4-(tridecanoylamino)butanoyl)-GLP-2(1-33);
L17K((S)-4-carboxy-4-(tetradecanoylamino)butanoyl)-GLP-2(1-33);
L17K((S)-4-carboxy-4-(pentadecanoylamino)butanoyl)-GLP-2(1-33);
L17K((S)-4-carboxy-4-(hexadecanoylamino)butanoyl)-GLP-2(1-33);
L17K((S)-4-carboxy-4-(heptadecanoylamino)butanoyl)-GLP-2(1-33);
L17K((S)-4-carboxy-4-(octadecanoylamino)butanoyl)-GLP-2(1-33);
L17K((S)-4-carboxy-4-(nonadecanoylamino)butanoyl)-GLP-2(1-33);
L17K((S)-4-carboxy-4-(eicosanoylamino)butanoyl)-GLP-2(1-33);
L17K(4-(octanoylamino)butanoyl)-GLP-2(1-33);
L17K(4-(nonanoylamino)butanoyl)-GLP-2(1-33);
L17K(4-(decanoylamino)butanoyl)-GLP-2(1-33);
L17K(4-(undecanoylamino)butanoyl)-GLP-2(1-33);
L17K(4-(dodecanoylamino)butanoyl)-GLP-2(1-33);
L17K(4-(tridecanoylamino)butanoyl)-GLP-2(1-33);
L17K(4-(tetradecanoylamino)butanoyl)-GLP-2(1-33);
L17K(4-(pentadecanoylamino)butanoyl)-GLP-2(1-33);
L17K(4-(hexadecanoylamino)butanoyl)-GLP-2(1-33);
L17K(4-(heptadecanoylamino)butanoyl)-GLP-2(1-33);
L17K(4-(octadecanoylamino)butanoyl)-GLP-2(1-33);
L17K(4-(nonadecanoylamino)butanoyl)-GLP-2(1-33);
L17K(4-(eicosanoylamino)butanoyl)-GLP-2(1-33);
A18K(3-(hexadecanoylamino)propionyl)-GLP-2(1-33);
D21K(3-(hexadecanoylamino)propionyl)-GLP-2(1-33);
N24K(3-(hexadecanoylamino)propionyl)-GLP-2(1-33);
Q28K(3-(hexadecanoylamino)propionyl)-GLP-2(1-33);
S5K(3-(hexadecanoylamino)propionyl)/K30R-GLP-2(1-33);
S7K(3-(hexadecanoylamino)propionyl)/K30R-GLP-2(1-33);
D8K(3-(hexadecanoylamino)propionyl)/K30R-GLP-2(1-33);
E9K(3-(hexadecanoylamino)propionyl)/K30R-GLP-2(1-33);
M10K(3-(hexadecanoylamino)propionyl)/K30R-GLP-2(1-33);
N11K(3-(hexadecanoylamino)propionyl)/K30R-GLP-2(1-33);
T12K(3-(hexadecanoylamino)propionyl)/K30R-GLP-2(1-33);
I13K(3-(hexadecanoylamino)propionyl)/K30R-GLP-2(1-33);
L14K(3-(hexadecanoylamino)propionyl)/K30R-GLP-2(1-33);
D15K(3-(hexadecanoylamino)propionyl)/K30R-GLP-2(1-33);
N16K(3-(hexadecanoylamino)propionyl)/K30R-GLP-2(1-33);
L17K(3-(octanoylamino)propionyl)/K30R-GLP-2(1-33);
L17K(3-(nonanoylamino)propionyl)/K30R-GLP-2(1-33);
L17K(3-(decanoylamino)propionyl)/K30R-GLP-2(1-33);
L17K(3-(undecanoylamino)propionyl)/K30R-GLP-2(1-33);
L17K(3-(dodecanoylamino)propionyl)/K30R-GLP-2(1-33);
L17K(3-(tridecanoylamino)propionyl)/K30R-GLP-2(1-33);
L17K(3-(tetradecanoylamino)propionyl)/K30R-GLP-2(1-33);
L17K(3-(pentadecanoylamino)propionyl)/K30R-GLP-2(1-33);
L17K(3-(hexadecanoylamino)propionyl)/K30R-GLP-2(1-33);
L17K(3-(heptadecanoylamino)propionyl)/K30R-GLP-2(1-33);
L17K(3-(octadecanoylamino)propionyl)/K30R-GLP-2(1-33);
L17K(3-(nonadecanoylamino)propionyl)/K30R-GLP-2(1-33);
L17K(3-(eicosanoylamino)propionyl)/K30R-GLP-2(1-33);
L17K((S)-4-carboxy-4-(octanoylamino)butanoyl)/K30R-GLP-2(1-33);
L17K((S)-4-carboxy-4-(nonanoylamino)butanoyl)/K30R-GLP-2(1-33);
L17K((S)-4-carboxy-4-(decanoylamino)butanoyl)/K30R-GLP-2(1-33);
L17K((S)-4-carboxy-4-(undecanoylamino)butanoyl)/K30R-GLP-2(1-33);
L17K((S)-4-carboxy-4-(dodecanoylamino)butanoyl)/K30R-GLP-2(1-33);
L17K((S)-4-carboxy-4-(tridecanoylamino)butanoyl)/K30R-GLP-2(1-33);
L17K((S)-4-carboxy-4-(tetradecanoylamino)butanoyl)/K30R-GLP-2(1-33);
L17K((S)-4-carboxy-4-(pentadecanoylamino)butanoyl)/K30R-GLP-2(1-33);
L17K((S)-4-carboxy-4-(hexadecanoylamino)butanoyl)/K30R-GLP-2(1-33);
L17K((S)-4-carboxy-4-(heptadecanoylamino)butanoyl)/K30R-GLP-2(1-33);
L17K((S)-4-carboxy-4-(octadecanoylamino)butanoyl)/K30R-GLP-2(1-33);
L17K((S)-4-carboxy-4-(nonadecanoylamino)butanoyl)/K30R-GLP-2(1-33);
L17K((S)-4-carboxy-4-(eicosanoylamino)butanoyl)/K30R-GLP-2(1-33);
L17K(4-(octanoylamino)butanoyl)/K30R-GLP-2(1-33);
L17K(4-(nonanoylamino)butanoyl)/K30R-GLP-2(1-33);
L17K(4-(decanoylamino)butanoyl)/K30R-GLP-2(1-33);
L17K(4-(undecanoylamino)butanoyl)/K30R-GLP-2(1-33);
L17K(4-(dodecanoylamino)butanoyl)/K30R-GLP-2(1-33);
L17K(4-(tridecanoylamino)butanoyl)/K30R-GLP-2(1-33);
L17K(4-(tetradecanoylamino)butanoyl)/K30R-GLP-2(1-33);
L17K(4-(pentadecanoylamino)butanoyl)/K30R-GLP-2(1-33);
L17K(4-(hexadecanoylamino)butanoyl)/K30R-GLP-2(1-33);
L17K(4-(heptadecanoylamino)butanoyl)/K30R-GLP-2(1-33);
L17K(4-(octadecanoylamino)butanoyl)/K30R-GLP-2(1-33);
L17K(4-(nonadecanoylamino)butanoyl)/K30R-GLP-2(1-33);
L17K(4-(eicosanoylamino)butanoyl)/K30R-GLP-2(1-33);
A18K(3-(hexadecanoylamino)propionyl)/K30R-GLP-2(1-33);
D21K(3-(hexadecanoylamino)propionyl)/K30R-GLP-2(1-33);
N24K(3-(hexadecanoylamino)propionyl)/K30R-GLP-2(1-33);
Q28K(3-(hexadecanoylamino)propionyl)/K30R-GLP-2(1-33);
D3E/S5K(3-(hexadecanoylamino)propionyl)/K30R/D33E-GLP-2(1-33);
D3E/S7K(3-(hexadecanoylamino)propionyl)/K30R/D33E-GLP-2(1-33);
D3E/D8K(3-(hexadecanoylamino)propionyl)/K30R/D33E-GLP-2(1-33);
D3E/E9K(3-(hexadecanoylamino)propionyl)/K30R/D33E-GLP-2(1-33);
D3E/M10K(3-(hexadecanoylamino)propionyl)/K30R/D33E-GLP-2(1-33);
D3E/N11K(3-(hexadecanoylamino)propionyl)/K30R/D33E-GLP-2(1-33);
D3E/T12K(3-(hexadecanoylamino)propionyl)/K30R/D33E-GLP-2(1-33);
D3E/I13K(3-(hexadecanoylamino)propionyl)/K30R/D33E-GLP-2(1-33);
D3E/L14K(3-(hexadecanoylamino)propionyl)/K30R/D33E-GLP-2(1-33);
D3E/D15K(3-(hexadecanoylamino)propionyl)/K30R/D33E-GLP-2(1-33);
D3E/N16K(3-(hexadecanoylamino)propionyl)/K30R/D33E-GLP-2(1-33);
D3E/L17K(3-(octanoylamino)propionyl)/K30R/D33E-GLP-2(1-33);
D3E/L17K(3-(nonanoylamino)propionyl)/K30R/D33E-GLP-2(1-33);
D3E/L17K(3-(decanoylamino)propionyl)/K30R/D33E-GLP-2(1-33);
D3E/L17K(3-(undecanoylamino)propionyl)/K30R/D33E-GLP-2(1-33);
D3E/L17K(3-(dodecanoylamino)propionyl)/K30R/D33E-GLP-2(1-33);
D3E/L17K(3-(tridecanoylamino)propionyl)/K30R/D33 E-GLP-2(1-33);
D3E/L17K(3-(tetradecanoylamino)propionyl)/K30R/D33E-GLP-2(1-33);
D3E/L17K(3-(pentadecanoylamino)propionyl)/K30R/D33E-GLP-2(1-33);
D3E/L17K(3-(hexadecanoylamino)propionyl)/K30R/D33E-GLP-2(1-33);
D3E/L17K(3-(heptadecanoylamino)propionyl)/K30R/D33E-GLP-2(1-33);
D3E/L17K(3-(octadecanoylamino)propionyl)/K30R/D33E-GLP-2(1-33);
D3E/L17K(3-(nonadecanoylamino)propionyl)/K30R/D33E-GLP-2(1-33);
D3E/L17K(3-(eicosanoylamino)propionyl)/K30R/D33E-GLP-2(1-33);
D3E/L17K((S)-4-carboxy-4-(octanoylamino)butanoyl)/K30R/D33E-GLP-2(1-33);
D3E/L17K((S)-4-carboxy-4-(nonanoylamino)butanoyl)/K30R/D33E-GLP-2(1-33);
D3E/L17K((S)-4-carboxy-4-(decanoylamino)butanoyl)/K30R/D33E-GLP-2(1-33);
D3E/L17K((S)-4-carboxy-4-(undecanoylamino)butanoyl)/K30R/D33E-GLP-2(1-33);
D3E/L17K((S)-4-carboxy-4-(dodecanoylamino)butanoyl)/K30R/D33E-GLP-2(1-33);
D3E/L17K((S)-4-carboxy-4-(tridecanoylamino)butanoyl)/K30R/D33E-GLP-2(1-33);
D3E/L17K((S)-4-carboxy-4-(tetradecanoylamino)butanoyl)/K30R/D33E-GLP-2(1-33);
D3E/L17K((S)-4-carboxy-4-(pentadecanoylamino)butanoyl)/K30R/D33E-GLP-2(1-33);
D3E/L17K((S)-4-carboxy-4-(hexadecanoylamino)butanoyl)/K30R/D33E-GLP-2(1-33);
D3E/L17K((S)-4-carboxy-4-(heptadecanoylamino)butanoyl)/K30R/D33E-GLP-2(1-33);
D3E/L17K((S)-4-carboxy-4-(octadecanoylamino)butanoyl)/K30R/D33E-GLP-2(1-33);
D3E/L17K((S)-4-carboxy-4-(nonadecanoylamino)butanoyl)/K30R/D33E-GLP-2(1-33);
D3E/L17K((S)-4-carboxy-4-(eicosanoylamino)butanoyl)/K30R/D33E-GLP-2(1-33);
D3E/L17K(4-(octanoylamino)butanoyl)/K30R/D33E-GLP-2(1-33);
D3E/L17K(4-(nonanoylamino)butanoyl)/K30R/D33E-GLP-2(1-33);
D3E/L17K(4-(decanoylamino)butanoyl)/K30R/D33E-GLP-2(1-33);
D3E/L17K(4-(undecanoylamino)butanoyl)/K30R/D33E-GLP-2(1-33);
D3E/L17K(4-(dodecanoylamino)butanoyl)/K30R/D33E-GLP-2(1-33);
D3E/L17K(4-(tridecanoylamino)butanoyl)/K30R/D33E-GLP-2(1-33);
D3E/L17K(4-(tetradecanoylamino)butanoyl)/K30R/D33E-GLP-2(1-33);
D3E/L17K(4-(pentadecanoylamino)butanoyl)/K30R/D33E-GLP-2(1-33);
D3E/L17K(4-(hexadecanoylamino)butanoyl)/K30R/D33E-GLP-2(1-33);
D3E/L17K(4-(heptadecanoylamino)butanoyl)/K30R/D33E-GLP-2(1-33);
D3E/L17K(4-(octadecanoylamino)butanoyl)/K30R/D33E-GLP-2(1-33);
D3E/L17K(4-(nonadecanoylamino)butanoyl)/K30R/D33E-GLP-2(1-33);
D3E/L17K(4-(eicosanoylamino)butanoyl)/K30R/D33E-GLP-2(1-33);
D3E/A18K(3-(hexadecanoylamino)propionyl)/K30R/D33E-GLP-2(1-33);
D3E/D21K(3-(hexadecanoylamino)propionyl)/K30R/D33E-GLP-2(1-33);
D3E/N24K(3-(hexadecanoylamino)propionyl)/K30R/D33E-GLP-2(1-33); and
D3E/Q28K(3-(hexadecanoylamino)propionyl)/K30R/D33E-GLP-2(1-33). A GLP-2 compound can contain any suitable combination of such GLP-2 derivatives and/or other GLP-2 compounds (including other GLP-2 derivatives, GLP-2 analogs, and/or GLP-2 peptides).

In a further aspect, the present invention relates to (in the context of the methods and/or compositions of the invention) a GLP-2 derivative in which the C-terminal amino acid residue is present in the form of the amide.

In a further aspect, the present invention relates to a GLP-2 derivative having a lipophilic substituent which can be negatively charged. In one aspect the group which can be negatively charged is a carboxylic acid group.

The GLP-2 compound is preferably a GLP-2 peptide, a nucleic acid encoding a GLP-2 peptide, a vector comprising such a nucleic acid, or a host cell comprising such a nucleic acid or vector. GLP-2 peptides, per se (e.g., GLP-2 derivatives), are particularly preferred GLP-2 compounds in most method and composition aspects of the invention.

GLP-2 peptides can be produced by any suitable method. For example, a GLP-2 peptide (or parent GLP-2 peptide in the case of a GLP-2 derivative) can be prepared by a method which comprises culturing a host cell containing a DNA sequence encoding the GLP-2 peptide and capable of expressing the GLP-2 peptide in a suitable nutrient medium under conditions permitting the expression of the GLP-2 peptide, after which the resulting GLP-2 peptide is recovered from the culture.

The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The GLP-2 peptide produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. ion exchange chromatography, gel filtration chromatography, affinity chromatography, or the like, dependent on the type of GLP-2 peptide in question.

A nucleic acid sequence encoding the GLP-2 peptide can be any suitable sequence, such as a genomic DNA sequence or cDNA sequence encoding a GLP-2 peptide, which, for instance, can be obtained by preparing a genomic or cDNA library and screening for DNA sequences coding for all or part of the desired GLP-2 peptide by hybridization using synthetic oligonucleotide probes in accordance with standard techniques (see, for example, Sambrook, J, Fritsch, E F and Maniatis, T, Molecular Cloning: A Laboratory Manual, Cold Spring Harbor Laboratory Press, New York, 1989). A DNA or other nucleic acid sequence encoding the GLP-2 peptide may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. A DNA sequence also may also be prepared by polymerase chain reaction using specific primers, for instance as described in U.S. Pat. No. 4,683,202 or Saiki et al., Science 239 (1988), 487-491.

A GLP-2 encoding DNA sequence or other suitable GLP-2 encoding nucleic acid sequence may be inserted into any suitable vector (preferably which may conveniently be subjected to recombinant DNA procedures). The choice of vector often depends on the host cell into which it is to be introduced. Thus, the vector may be an autonomously replicating vector, i.e. a vector which exists as an extrachromosomal entity, the replication of which typically is independent of chromosomal replication, e.g. a plasmid. Alternatively, the vector may be one which, when introduced into a host cell, is integrated into the host cell genome and replicated together with the chromosome(s) into which it has been integrated.

A vector comprising a GLP-2 peptide-encoding nucleic acid sequence is preferably an expression vector in which a DNA sequence encoding a GLP-2 peptide is operably linked to additional sequences/segments useful in or required for transcription of the GLP-2 peptide-encoding sequence, such as a promoter, an enhancer, a Kozak consensus sequence, etc. A promoter can be any DNA sequence which shows transcriptional activity in the host cell of choice and may be derived from genes encoding proteins either homologous or heterologous to the host cell. Examples of suitable promoters for directing the transcription of the DNA encoding the GLP-2 peptide of the invention in a variety of host cells are well known in the art, cf. for instance Sambrook et al., supra.

A DNA sequence encoding the GLP-2 peptide may also, if necessary, be operably connected to a suitable terminator, polyadenylation signal(s), transcriptional enhancer sequences, and translational enhancer sequences. A recombinant vector of the invention may further comprise a DNA sequence enabling the vector to replicate in the host cell in question.

A vector may also comprise a selectable marker, e.g. a gene the product of which complements a defect in the host cell or one which confers resistance to a drug, e.g. ampicillin, kanamycin, tetracycline, chloramphenicol, neomycin, hygromycin, and/or methotrexate.

To facilitate directing the GLP-2 peptide (e.g., a parent GLP-2 peptide intermediate in the production of a GLP-2 derivative) into the secretory pathway of the host cells, a secretory signal sequence (also known as a leader sequence, prepro sequence or pre sequence) may be provided in the recombinant vector. A secretory signal sequence typically is included in a GLP-2 peptide-encoding DNA in the correct reading frame and located 5′ to the DNA sequence encoding the GLP-2 peptide. A secretory signal sequence may be a sequence that is normally associated with a GLP-2 peptide or may correspond to another (non-GLP-2) secreted protein.

The procedures used to ligate DNA peptide-coding sequences (e.g., a sequence coding for one or more GLP-2 peptides), promoters, terminators and/or secretory signal sequences, respectively, and to insert them into suitable vectors containing the information necessary for replication, are well known to persons skilled in the art (cf., for instance, Sambrook et al., supra).

The host cell into which the DNA sequence or the recombinant vector is introduced may be any cell which is capable of producing the present GLP-2 peptides, such as bacteria, yeast, fungi and higher eukaryotic cells. Examples of suitable host cells well known and used in the art are, without limitation, E. coli, Saccharomyces cerevisiae, and mammalian BHK or CHO cell lines.

GLP-2 derivatives of the invention can be prepared by introducing the lipophilic substituent into the parent GLP-2 peptide using methods known per se, see for example WO 95/07931, the contents of which is hereby incorporated in its entirety by reference.

N^{ε}-acylation of a Lys residue can be carried out by using an activated amide of the acyl group to be introduced as the acylating agent, e.g. the amide with benzotriazole. The acylation can be carried out in a polar solvent in the presence of a base.

Pharmaceutical compositions containing a GLP-2 compound can be administered by any suitable route of administration and in any suitable form, a number examples of which are provided elsewhere herein.

In certain aspects, a GLP-2 compound composition is administered parenterally to patients in need of treatment with the GLP-2 compound. Parenteral administration may be performed by subcutaneous, intramuscular or intravenous injection by means of a syringe, optionally a pen-like syringe. Alternatively, parenteral administration can be performed by means of an infusion pump. GLP-2 compound compositions formulated for such forms of delivery are important aspects of the invention.

In another aspect, the invention provides a pharmaceutically acceptable composition comprising a GLP-2 compound (in an amount sufficient to promote, induce, and/or enhance a desired physiological effect in a mammal, wherein the composition is in the form of a powder or a liquid for the administration nasal or pulmonary administration (e.g., as a nasal spray).

In other aspects, the invention provides a GLP-2 compound composition that is formulated for transdermal administration. For example, in one aspect the invention provides a GLP-2 compound composition that is contained in a drug-in-matrix, drug-in-reservoir, iontophoretic, or other suitable type of “patch” for transmission across the skin. In other aspects, the invention provides a GLP-2 compound composition that is formulated for buccal and/or transmucosal administration. A composition for nasal administration of GLP-2 may, for example, be prepared as described in European Patent No. 272097 (to Novo Nordisk A/S) or in WO 93/18785.

For the most part, pharmaceutically acceptable formulations containing a GLP-2 compound for administration to a patient may be prepared by conventional techniques, e.g. as described in Remington's Pharmaceutical Sciences, 1985 or in Remington: The Science and Practice of Pharmacy, 19th edition, 1995. Thus, for example, an injectable composition of a GLP-2 compound can be prepared using the conventional technique of dissolving and mixing the ingredients of the composition, as appropriate, to obtain a desired end product composition. Thus, according to one exemplary procedure, a GLP-2 compound is dissolved in an amount of water which is somewhat less than the final volume of the composition to be prepared. An isotonic agent, a preservative, and a buffer are added as desired and the pH value of the solution is adjusted—if necessary—using an acid, e.g. hydrochloric acid, or a base, e.g. aqueous sodium hydroxide as desired. Finally, the volume of the solution is adjusted with water to give the desired concentration of the ingredients. Examples of isotonic agents are sodium chloride, mannitol and glycerol. Examples of preservatives are phenol, m-cresol, methyl p-hydroxybenzoate, and benzyl alcohol. Examples of suitable buffers are sodium acetate and sodium phosphate. Further to the above-mentioned components, solutions containing a GLP-2 compound according to the present invention may also contain a surfactant in order to improve the solubility and/or the stability of the receptor agonist.

The pharmacological properties of the compounds of the invention can be tested e.g. as described in International Patent Application No. PCT/DK97/00086, WO 97/31943.

In a further aspect the invention relates to a pharmaceutical formulation comprising a GLP-2 compound and a buffer, wherein the GLP-2 compound is present in a concentration from about 0.001 mg/ml to about 0.1 mg/ml (e.g., about 0.05-0.1 mg/ml, about 0.005-0.1 mg/ml, about 0.01-0.1 mg/ml, etc.), and wherein the formulation has a pH from about 8 to about 10 (e.g., about 8.5-9.5).

In a further aspect, the invention relates to a pharmaceutical formulation comprising an aqueous solution of a GLP-2 compound and a buffer, wherein the GLP-2 compound is present in a concentration from about 0.001 mg/ml to about 0.1 mg/ml (e.g., about 0.05-0.1 mg/ml, about 0.005-0.1 mg/ml, or about 0.01-0.1 mg/ml), and wherein the formulation has a pH from about 8.0 to about 10 (e.g., about 8.5-9.5).

In a further aspect the invention relates to a method of preparing a physically stable pharmaceutical formulation of a GLP-2 compound comprising preparing a formulation containing the GLP-2 compound, and a buffer, wherein the GLP-2 compound is present in a concentration from about 0.001 mg/ml to about 0.1 mg/ml, and wherein the formulation has a pH from about 8.0 to about 10.

In a further aspect the invention relates to a method of preparing a physically stable pharmaceutical formulation of a GLP-2 compound comprising preparing an aqueous solution containing the GLP-2 compound, and a buffer, wherein the GLP-2 compound is present in a concentration from about 0.001 mg/ml to about 0.1 mg/ml, and wherein the formulation has a pH from about 8.0 to about 10.

In a further aspect the invention relates to a method of preparing a physically stable pharmaceutical formulation of a GLP-2 compound comprising preparing a formulation containing the GLP-2 compound, water, and a buffer, wherein the GLP-2 compound is present in a concentration from about 0.001 mg/ml to about 0.1 mg/ml, and wherein the formulation has a pH from about 8.0 to about 10.

In another aspect, the invention provides a method for inducing, promoting, and/or enhancing one or more physiological responses in a mammal (e.g., a human patient) associated with the treatment or prevention of intestinal failure or other condition leading to malabsorption of nutrients in the intestine, comprising administering to a patient in need thereof an effective amount of a pharmaceutically acceptable composition comprising an aqueous solution of a GLP-2 compound, and a buffer, wherein the GLP-2 compound is present in a concentration from about 0.001 mg/ml to about 0.1 mg/ml (e.g., about 0.05-0.1 mg/ml, about 0.005-0.1 mg/ml, about 0.001-0.01 mg/ml, about 0.005-0.01 mg/ml, about 0.001-0.05 mg/ml, etc.).

The term “an effective amount” refers to a dose determined to be effective by a qualified practitioner, who may titrate dosages to achieve the desired response, body of practitioners, and/or regulatory agency (the latter two cases typically being determined for a population of patients having a similar set of conditions). Factors for consideration of dose can include potency, bioavailability, desired pharmacokinetic/pharmacodynamic profiles, condition of treatment (e.g. diabetes, obesity, weight loss, gastric ulcers, Crohn's disease, GERD, etc.), patient-related factors (e.g. weight, health, age, etc.), presence of co-administered medications (e.g., insulin, an EGF, a keratinocyte growth factor (KGF—such as KGF-2 or an analog or derivative thereof), a growth hormone (e.g., hGH), an insulin-like growth factor (e.g., IGF-1 and/or IGF-2), a vascular endothelial growth factor (VEGF), a fibroblast growth factor (FGF), etc. (and related nucleic acids, vectors, and cells) each of which and/or combinations thereof can be suitable additional medicaments for administration in methods described herein), time of administration, or other factors known to medical practitioners.

Also some nutrients such as glutamine, arginine, omega-3 fatty acids, and probiotics have been shown to influence intestinal barrier function and immune system and it is therefore an aspect of the present invention to treat ischemia-reperfusion injuries with a GLP-2 compound in combination with nutritional supplements by enteral or parenteral intake.

In a further aspect, the invention relates to a method for the treatment of ischemia-reperfusion injury in a subject comprising administering to said subject a composition comprising a therapeutically or prophylactically effective amount of a GLP-2 compound, whereby at least one symptom of ischemia-reperfusion injury is alleviated, wherein said composition contain a further compound selected from the group consisting of Insulin-like Growth Factor 1 (IGF-1), analogs of IGF-1, Insulin-like Growth Factor 2 (IGF-2), analogs of IGF-2, Growth Hormone (GH), analogs of GH and IL-10.

In certain aspects, a GLP-2 compound (such as a GLP-2 peptide—e.g., a GLP-2 peptide derivative) is administered in the form of an aqueous pharmaceutically acceptable formulation—i.e. a formulation comprising water (such formulations also are, in and of themselves, important aspects of the invention). Such a formulation is typically a solution or a suspension. In a further aspect of the invention the pharmaceutical formulation is an aqueous solution. The term “aqueous formulation” is defined as a formulation comprising at least 50% w/w water. The term “aqueous solution” is defined as a solution comprising at least 50% w/w water, and the term “aqueous suspension” is defined as a suspension comprising at least 50% w/w water.

A pharmaceutical formulation of a GLP-2 compound also can be in the form a freeze-dried formulation, whereto the physician or the patient adds a solvent prior to administration. In another aspect the invention provides a pharmaceutical formulation comprising a GLP-2 compound that is a lyophilized formulation whereto the physician or the patient adds a solvent prior to use.

A pharmaceutical formulation is found to be physically unstable when it exhibits turbidity. Stable GLP-2 compound formulations of the invention may be physically stable for more than about 3 months, more than about 6 months, more than about 9 months, more than about 10 months, more than about 11 months, more than about one year, more than about 18 months, more than about 22 months, or even more than about two years at temperatures of about 5° C. or less.

Physical stability of the formulations can be evaluated by means of visual inspection and evaluating turbidity after storage of the formulation at different temperatures in top filled glass cartridges for various time periods. Visual inspection of the formulations can be performed, for example, in a sharp focused light with a dark background. The turbidity of the formulation can be characterized by a visual score ranking the degree of turbidity from 0 to about 3 (a formulation showing no turbidity corresponds to a visual score of 0, and a formulation showing visual turbidity in daylight corresponds to visual score of 3). A formulation can be classified as physical unstable with respect to protein aggregation, when it shows visual turbidity in daylight.

GLP-2 compound pharmaceutical formulations of the invention advantageously can remain physically stable for at least about 6 weeks, at least about 9 weeks, more than about 12 weeks, more than about 6 months, more than about one year, more than about 15 months, or longer at temperatures of about 5° C. or less, as measured by stability tests known to the person skilled in the art and as approved by regulatory authorities. Stability method references that may be used as guides for analytical method validation include “Stability of Pharmaceutical Products” Remington's Pharmaceutical Sciences, 1985. It is appropriate to note that some specific stability tests are often indicated for certain pharmaceutical dosage forms in addition to those normally conducted.

GLP-2 compound formulations of the invention also advantageously retain physical stability at higher temperatures. For example, GLP-2 formulations of the invention can retain physical stability for about 6 weeks or longer, about 9 weeks or longer, about 12 weeks or longer at temperatures of about 25° C. or less (e.g., temperatures of about 5-25° C., temperatures of about 10-25° C., temperatures of about 15-25° C., or temperatures of about 20-25° C.).

It has been found that the pH of a GLP-2 compound formulation can impact the usefulness of the formulation in treatment. Thus, for example, in one aspect the invention provides a GLP-2 formulation has a pH in the range from about 7.6 to about 10; in another aspect the GLP-2 composition has a pH of about 7.7 to about 10; in a further aspect the invention provides a formulation has a pH in the range from about 7.8 to about 10. In yet another aspect, the invention provides a formulation having a pH of about 7.9 to about 10. In additional aspects, the invention provides GLP-2 compound formulations having a pH of about 8.0 to about 10, about 8.0 to about 9.5, about 8 to about 9, about 8 to about 8.5, about 8.5 to about 10, about 8.5 to about 9.5, about 8.5 to about 9, about 9 to about 10, about 9 to about 9.5, or about 9.5 to about 10.

In a further aspect of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, N-2-Hydroxyethylpiperazine-N′-2-ethanesulfonic acid (HEPES), N,N-bis-(2-hydroxyethyl)-glycin (BICIN), Diammonium hydrogen phosphate ((NH4)2HPO4) and tris(hydroxymethyl)-aminomethan, or mixtures thereof. Each one of these specific buffers constitutes an alternative aspect of the invention. In a further aspect of the invention the buffer is glycylglycine, sodium dihydrogen phosphate, disodium hydrogen phosphate, sodium phosphate or mixtures of any or all thereof.

In another aspect, the invention provides a GLP-2 compound formulation that also or alternatively comprises a pharmaceutically acceptable preservative. GLP-2 compound formulations can include any suitable preservative. Examples of preservatives include phenol, m-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, or mixtures of any or all thereof. Formulations comprising each of these specific preservatives constitutes an alternative aspect of the invention. In a preferred aspect of the invention the preservative is phenol or m-cresol.

Where the invention provides a GLP-2 compound formulation comprising a preservative, the preservative can be present in any suitable amount. Typically, in such formulations, the preservative component is present in a concentration from 0.1 mg/ml to 20 mg/ml. In a further aspect of the invention the preservative is present in a concentration from 0.1 mg/ml to 5 mg/ml. In a further aspect of the invention the preservative is present in a concentration from 5 mg/ml to 10 mg/ml. In a further aspect of the invention the preservative is present in a concentration from 10 mg/ml to 20 mg/ml. Formulations comprising each one of these specific concentration ranges constitute alternative aspects of the invention.

The use of a preservative in pharmaceutical compositions is well-known to the skilled person. For convenience reference is made to Remington: The Science and Practice of Pharmacy, 19th edition, 1995.

In a further aspect of the invention the formulation further comprises an isotonic agent. In a further aspect of the invention the isotonic agent is selected from the group consisting of a salt (e.g. sodium chloride), a polyhydric alcohol (e.g. propyleneglycol, xylitol, mannitol, sorbitol or glycerol), a monosaccharide (e.g. glucose or maltose), a disccharide (e.g. sucrose), an amino acid (e.g. L-glycine, L-histidine, arginine, lysine, isoleucine, aspartic acid, tryptophan, threonine), polyethyleneglycol (e.g. PEG400), or mixtures thereof. In a further aspect of the invention the isotonic agent is selected from the group consisting of sodium chloride, glycerol, mannitol, glucose, sucrose, L-glycine, L-histidine, arginine, lysine or mixtures thereof. Each one of these specific isotonic agents constitutes an alternative aspect of the invention. In one aspects of the invention the isotonic agent is mannitol. In one aspects of the invention the isotonic agent is glycerol. In one aspects of the invention the isotonic agent is sucrose.

In a further aspect of the invention the isotonic agent is present in a concentration from about 1 mg/ml to about 50 mg/ml. In a further aspect of the invention the isotonic agent is present in a concentration from about 1 mg/ml to about 7 mg/ml. In a further aspect of the invention the isotonic agent is present in a concentration from about 8 mg/ml to about 16 mg/ml. In a further aspect of the invention the isotonic agent is present in a concentration from about 17 mg/ml to about 50 mg/ml. Each one of these specific concentration ranges constitutes an alternative aspect of the invention.

The use of an isotonic agent in pharmaceutical compositions is well-known to the skilled person. For convenience reference is made to Remington: The Science and Practice of Pharmacy, 19th edition, 1995.

In a further aspect of the invention the formulation further comprises a chelating agent. Any suitable chelating agent can be included in such formulations. In one exemplary aspect, the chelating agent is selected from salts of ethylenediaminetetraacetic acid (EDTA), citric acid, and aspartic acid, and mixtures thereof. Each one of these specific chelating agents constitutes an alternative aspect of the invention.

When present in a formulation of the invention, the chelating agent can be present in any suitable amount. In an exemplary aspect, the chelating agent is present in a concentration from about 0.1 mg/ml to about 5 mg/ml. In a further aspect of the invention the chelating agent is present in a concentration from about 0.1 mg/ml to about 2 mg/ml. In a further aspect of the invention the chelating agent is present in a concentration from about 2 mg/ml to about 5 mg/ml.

The use of a chelating agent in pharmaceutical compositions is well-known to the skilled person. For convenience reference is made to Remington: The Science and Practice of Pharmacy, 19th edition, 1995.

In a further aspect of the invention the formulation further comprises a stabilizer selected from the group of high molecular weight polymers or low molecular compounds. In a further aspect of the invention the stabilizer is selected from polyethylene glycol (e.g. PEG 3350), polyvinylalcohol (PVA), polyvinylpyrrolidone, carboxymethylcellulose, different salts (e.g. sodium chloride), L-glycine, L-histidine, imidazole, arginine, lysine, isoleucine, aspartic acid, tryptophan, threonine and mixtures thereof. Each one of these specific stabilizers constitutes an alternative aspect of the invention. In a preferred aspect of the invention the stabilizer is selected from the group consisting of L-histidine, imidazole and arginine.

In a further aspect of the invention a high molecular weight polymer is present in a concentration from about 0.1 mg/ml to about 50 mg/ml. In a further aspect of the invention the high molecular weight polymer is present in a concentration from about 0.1 mg/ml to about 5 mg/ml. In a further aspect of the invention the high molecular weight polymer is present in a concentration from about 5 mg/ml to about 10 mg/ml. In a further aspect of the invention the high molecular weight polymer is present in a concentration from about 10 mg/ml to about 20 mg/ml. In a further aspect of the invention the high molecular weight polymer is present in a concentration from about 20 mg/ml to about 30 mg/ml. In a further aspect of the invention the high molecular weight polymer is present in a concentration from about 30 mg/ml to about 50 mg/ml.

In a further aspect of the invention a low molecular weight compound is present in a concentration from about 0.1 mg/ml to about 50 mg/ml. In a further aspect of the invention the low molecular weight compound is present in a concentration from about 0.1 mg/ml to about 5 mg/ml. In a further aspect of the invention the low molecular weight compound is present in a concentration from about 5 mg/ml to about 10 mg/ml. In a further aspect of the invention the low molecular weight compound is present in a concentration from 10 mg/ml to 20 mg/ml. In a further aspect of the invention the low molecular weight compound is present in a concentration from about 20 mg/ml to about 30 mg/ml. In a further aspect of the invention the low molecular weight compound is present in a concentration from about 30 mg/ml to about 50 mg/ml.

The use of a stabilizer in pharmaceutical compositions is well-known to the skilled person. For convenience reference is made to Remington: The Science and Practice of Pharmacy, 19th edition, 1995.

In a further aspect of the invention the formulation further comprises a surfactant. In a further aspect of the invention the surfactant is selected from a detergent, ethoxylated castor oil, polyglycolyzed glycerides, acetylated monoglycerides, sorbitan fatty acid esters, poloxamers, such as 188 and 407, polyoxyethylene sorbitan fatty acid esters, polyoxyethylene derivatives such as alkylated and alkoxylated derivatives (tweens, e.g. Tween-20, or Tween-80), monoglycerides or ethoxylated derivatives thereof, diglycerides or polyoxyethylene derivatives thereof, glycerol, cholic acid or derivatives thereof, lecithins, alcohols and phospholipids, glycerophospholipids (lecithins, kephalins, phosphatidyl serine), glyceroglycolipids (galactopyransoide), sphingophospholipids (sphingomyelin), and sphingoglycolipids (ceramides, gangliosides), DSS (docusate sodium, CAS registry no [577-11-7]), docusate calcium, CAS registry no [128-49-4]), docusate potassium, CAS registry no [7491-09-0]), SDS (sodium dodecyl sulfate or sodium lauryl sulfate), dipalmitoyl phosphatidic acid, sodium caprylate, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, palmitoyl lysophosphatidyl-L-serine, lysophospholipids (e.g. 1-acyl-sn-glycero-3-phosphate esters of ethanolamine, choline, serine or threonine), alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, zwitterionic surfactants (e.g. N-alkyl-N,N-dimethylammonio-1-propanesulfonates, 3-cholamido-1-propyldimethylammonio-1-propanesulfonate, dodecylphosphocholine, myristoyl lysophosphatidylcholine, hen egg lysolecithin), cationic surfactants (quarternary ammonium bases) (e.g. cetyl-trimethylammonium bromide, cetylpyridinium chloride), non-ionic surfactants, polyethyleneoxide/polypropyleneoxide block copolymers (Pluronics/Tetronics, Triton X-100, Dodecyl β-D-glucopyranoside) or polymeric surfactants (Tween-40, Tween-80, Brij-35), fusidic acid derivatives- (e.g. sodium tauro-dihydrofusidate etc.), long-chain fatty acids and salts thereof C6-C12 (e.g. oleic acid and caprylic acid), acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, or the surfactant may be selected from the group of imidazoline derivatives, or mixtures thereof. Each one of these specific surfactants constitutes an alternative aspect of the invention.

The use of a surfactant in pharmaceutical compositions is well-known to the skilled person. For convenience reference is made to Remington: The Science and Practice of Pharmacy, 19th edition, 1995.

In one aspect of the invention the pharmaceutical formulation comprising the GLP-2 compound is free of one or more of the aforementioned excipients. For example, in one aspect, the invention provides pharmaceutically acceptable formulations consisting or consisting essentially of a GLP-2 compound in a sterile pyrogen-free solution comprising bacteriostatic water. The invention also provides a kit comprising such agents, which also may include assay materials for stability, toxicity, dose, etc.

In another aspect, the invention provides pharmaceutical formulations wherein the primary active ingredient, if not essentially only active ingredient, is a GLP-2 compound. In another aspect, the invention provides pharmaceutically acceptable formulations wherein a GLP-2 compound is the primary isolated active ingredient, if not the only detectable isolated active ingredient (e.g., the composition may contain unisolated growth hormone, epidermal growth factor, and the like in the form of a nutritional supplement).

In one exemplary aspect of the invention the pharmaceutical formulation comprising the GLP-2 compound is free of any presevative. In another exemplary aspect of the invention the pharmaceutical formulation comprising the GLP-2 compound is also or alternatively free of any stabilizer. In yet one more aspect of the invention the pharmaceutical formulation comprising the GLP-2 compound is also or alternatively free of any isotonic agent. In one aspect of the invention the pharmaceutical formulation comprising the GLP-2 compound is also or alternatively free of any chelating agent. In still another aspect of the invention the pharmaceutical formulation comprising the GLP-2 compound is also or alternatively free of any surfactant.

In another aspect, the invention provides a pharmaceutical formulation comprising an effective amount of a GLP-2 compound, wherein the formulation is free of any excipient. Such a composition may nonetheless include suitable diluents, such as sterile water for injection or other non-active carrier.

In another aspect, the invention relates to the preparation of a pharmaceutically acceptable composition comprising a GLP-2 compound having any one or combination of the above-described features.

The method typically comprises consulting a computer dosage program that considers the weight of the subject to be treated or analytical chart that provides the same functionality. The method also may optionally include testing the re-formulated composition for toxicity, stability, presence of contaminants, turbidity, and/or tonicity prior to administration. Such testing may be similarly provided by a device comprising a computer system for dosing, as mentioned above. Kits also including testing equipment, dosing equipment, and suitable diluents are provided by the invention. Such methods, kits, programs, and/or devices also can include means for assessing the dosage of GLP-2 compound in the formulation. In one aspect, the inventive method comprises formulating the GLP-2 compound with nutritional supplements and/or micronutrients. In another aspect, the inventive method also or alternatively comprises formulating the GLP-2 compound with a suitable anti-bacterial agent. In one aspect, the method comprises reconstituting a lyophylized GLP-2 composition comprising an adult dosage, as exemplified above. In another aspect, the inventive method also or alternatively comprises removing undesirable agents from the adult GLP-2 compound formulation, such as preservatives that may be potentially harmful. In a further aspect, the inventive method also or alternatively comprises storing the GLP-2 compound formulation in a sterile vial or ampuoule suitable for storage and rapid administration via a catheter, IV tube, or other device used to deliver pharmaceutical agents to a human subject. In another aspect, the invention provides a method for promoting the sale of suitable carriers for GLP-2 compounds, comprising distributing/disseminating information about the use of such carriers and/or kits comprising such carriers, for the preparation of GLP-2 compound formulations for the treatment of ischemia-reperfusion injury. Such information may include suitable doses for treating such conditions, stability information, re-formulation instructions, etc., and may be distributed to hospitals, pharmacists, formularies, primary care physicians, nurses, nurse practioners, insurance agencies, formularies, state and/or national regulatory agencies, etc., by means of internet advertising, phone advertising, e-mail marketing, direct mail marketing, hosting of seminars, providing free samples of such products, contacting key opinion leaders regarding such products through the use of liasons, funding publications describing the use of such products, etc.

In one aspect of the invention the pharmaceutical formulation comprising the GLP-2 compound is physically stable for more than 12 weeks and for more than 15 months at 5° C. as measured by visual inspection.

In another aspect of the invention the pharmaceutical formulation comprising the GLP-2 compound is physically stable for more than 12 weeks at 25° C. as measured by visual inspection.

In a further aspect of the invention the pharmaceutical formulation comprising the GLP-2 compound is physically stable for more than 12 weeks at 37° C. as measured by visual inspection.

In a further aspect of the invention the formulation comprising the GLP-2 compound has a pH in the range from 7.0 to 10. In a further aspect of the invention the formulation comprising the GLP-2 compound has a pH in the range from 7.0 to 9.5. In a further aspect of the invention the formulation comprising the GLP-2 compound has a pH in the range from 7.0 to 9.0. In a further aspect of the invention the formulation comprising the GLP-2 compound has a pH in the range from 7.0 to 8.5. In a further aspect of the invention the formulation comprising the GLP-2 compound has a pH in the range from 7.0 to 8.0. In a further aspect of the invention the formulation comprising the GLP-2 compound has a pH in the range from 7.0 to 7.5. In a further aspect of the invention the formulation comprising the GLP-2 compound has a pH in the range from 7.5 to 10. In a further aspect of the invention the formulation comprising the GLP-2 compound has a pH in the range from 8.0 to 10. In a further aspect of the invention the formulation comprising the GLP-2 compound has a pH in the range from 8.5 to 10. In a further aspect of the invention the formulation comprising the GLP-2 compound has a pH in the range from 9.0 to 10. In a further aspect of the invention the formulation comprising the GLP-2 compound has a pH in the range from 9.5 to 10. In a further aspect of the invention the formulation comprising the GLP-2 compound has a pH in the range from 7.5 to 9.5. In a further aspect of the invention the formulation comprising the GLP-2 compound has a pH in the range from 7.5 to 9.0. In a further aspect of the invention the formulation comprising the GLP-2 compound has a pH in the range from 7.5 to 8.5. In a further aspect of the invention the formulation comprising the GLP-2 compound has a pH in the range from 7.5 to 8.0. In a further aspect of the invention the formulation comprising the GLP-2 compound has a pH in the range from 8.0 to 9.5. In a further aspect of the invention the formulation comprising the GLP-2 compound has a pH in the range from 8.0 to 9.0. In a further aspect of the invention the formulation comprising the GLP-2 compound has a pH in the range from 8.0 to 8.5. In a further aspect of the invention the formulation comprising the GLP-2 compound has a pH in the range from 8.5 to 9.5. In a further aspect of the invention the formulation comprising the GLP-2 compound has a pH in the range from 8.5 to 9.0. In a further aspect of the invention the formulation comprising the GLP-2 compound has a pH in the range from 9.0 to 9.5.

In a further embodiment of the invention the GLP-2 compound is present in a concentration from 0.1 mg/ml to 100 mg/ml. In a further embodiment of the invention the GLP-2 compound is present in a concentration from 0.1 mg/ml to 80 mg/ml. In a further embodiment of the invention the GLP-2 compound is present in a concentration from 1 mg/ml to 80 mg/ml. In a further embodiment of the invention the GLP-2 compound is present in a concentration from 0.1 mg/ml to 50 mg/ml. In a further embodiment of the invention the GLP-2 compound is present in a concentration from 1 mg/ml to 50 mg/ml. In a further embodiment of the invention the GLP-2 compound is present in a concentration from 0.1 mg/ml to 20 mg/ml. In a further embodiment of the invention the GLP-2 compound is present in a concentration from 1 mg/ml to 20 mg/ml. In a further embodiment of the invention the GLP-2 compound is present in a concentration from 0.1 mg/ml to 10 mg/ml. In a further embodiment of the invention the GLP-2 compound is present in a concentration from 1 mg/ml to 10 mg/ml. In a further embodiment of the invention the GLP-2 compound is present in a concentration from 0.1-5 mg/ml. In a further embodiment of the invention the GLP-2 compound is present in a concentration from 1-5 mg/ml. In a further embodiment of the invention the GLP-2 compound is present in a concentration from 0.1-0.5 mg/ml. In a further embodiment of the invention the GLP-2 compound is present in a concentration from 0.6-1 mg/ml. Each one of these specific concentration ranges constitutes an alternative embodiment of the invention.

These foregoing features and aspects of the compositions/formulations of the invention can be combined in any suitable manner.

### BRIEF DESCRIPTION OF THE DRAWINGS

The present invention is described in further detail in the examples with reference to the appended drawings, which are as follows:

FIG. 1 The amino acid sequence of the 33 residues human GLP-2. The N-terminal His-Ala indicates the sequence cleaved of aminopeptidase dipeptidyl peptidase IV during metabolism of GLP-2. The Arg20 and Lys30 residues are the two basic amino acid residues in GLP-2.

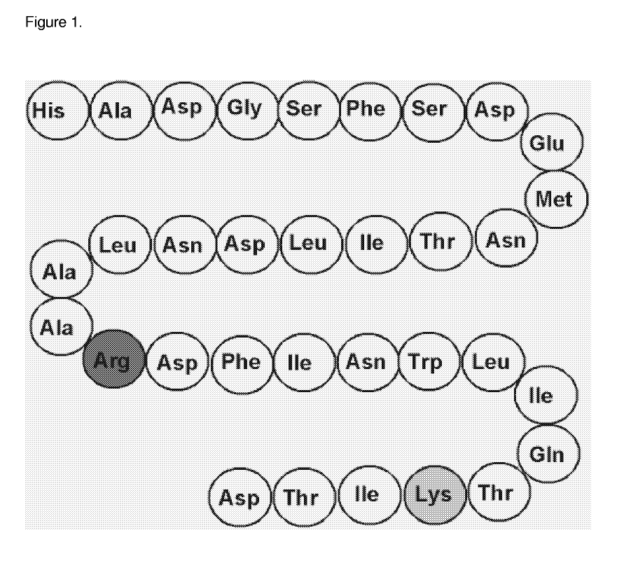

FIG. 2 L17K/K30R-GLP-2 (1-33) acylated with β-alanine C16.

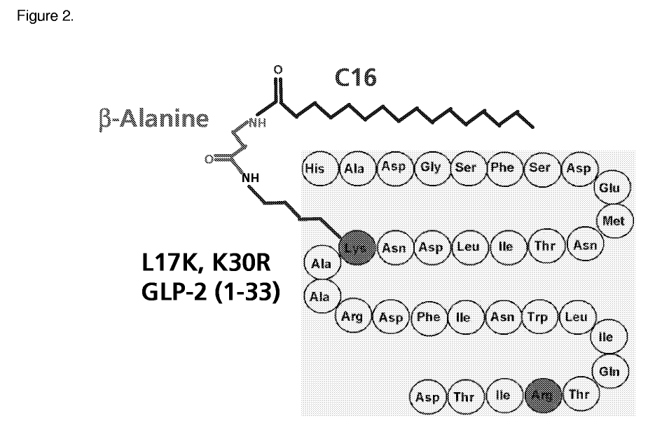

FIG. 3 Examples with chemical structure of the use of different spacers according to the invention with the lipophilic substituent being a hexadecanoyl.

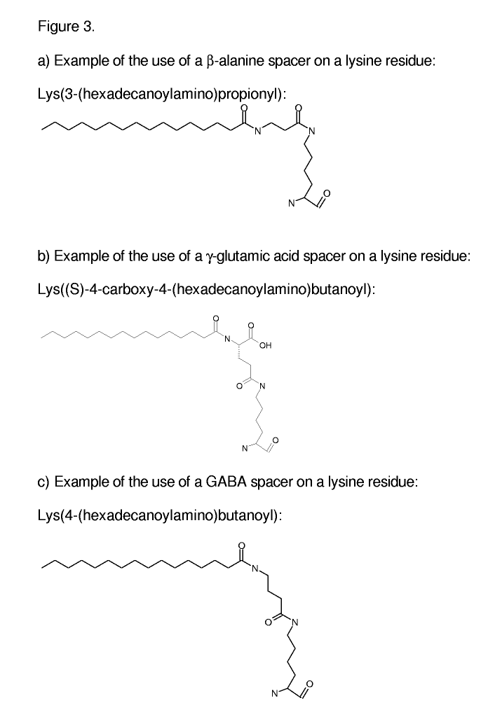

FIG. 4. SEQ ID NO. 1, SEQ ID NO. 2, and SEQ ID NO. 3.

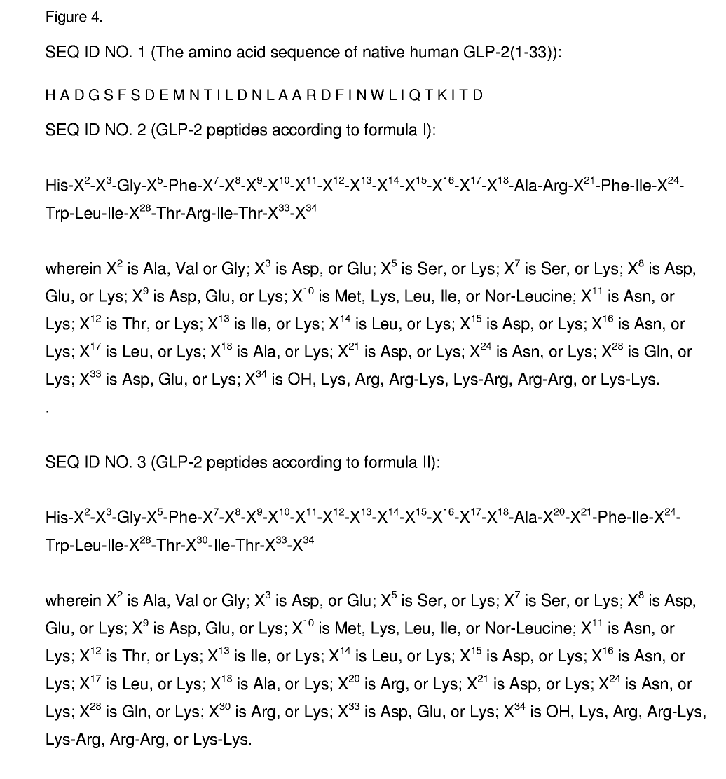

FIG. 5. Correlation between time (h) and IL-6 concentration (pg/ml) in plasma after mesenteric ischemia-reperfusion (I/R) with and without pretreatment with GLP-2 compound. MPO values on the right indicates filtration of neutrophiles in the intestine at the end of the experiment (4).

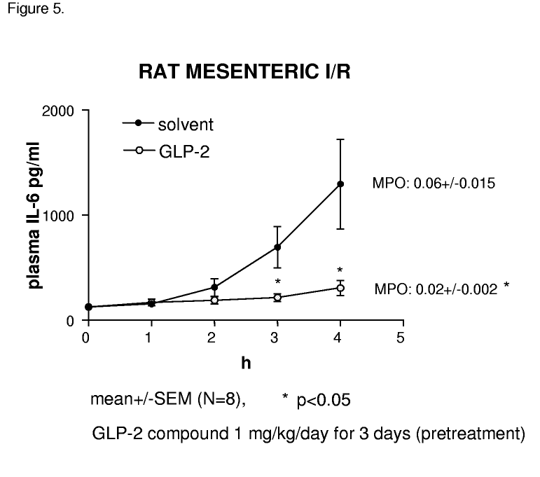

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

### EXAMPLES

### Example 1

### The protective Effect of GLP-2 Pre-Treatment on Mesenteric Ischemic Damages

The present study was initiated in order to investigate 1): the capacity of a protease-resistant GLP-2 compound to inhibit acute intestinal damages caused by mesenteric ischemia-reperfusion (I/R), and 2): to investigate if the latter effect involves secondary protein synthesis.

Methods: Control or GLP-2 compound pre-treated pentobarbital anaesthetized rats were laperatomised and the mesenteric artery was exposed. After 45 min of arterial constriction the clamp was released and the intestinal reperfusion was followed for additional 4 hours. Plasma IL-6 was measured repeatedly and at the end neutrophil accumulation was quantified by MPO-activity in a segment of jejunum and in lung.

Results: Mesenteric I/R was followed by marked and 5-10 fold elevations of plasma IL-6 and at the end of experiment intestine and lunge PMN-accumulations were evidenced by approximately doubling of the MPO-activities in the tissues. The GLP-2 compound administered 1 mg/kg sc for three days prior to experiment, in contrast to acutely administered compound, almost complete abolished the pathological manifestations. This protective effect of the GLP-2 compound was antagonised by a single co-administration of cycloheximide 1.2 mg/kg ip at 24 hour before experiment.

Conclusion: We have shown that pre-treatment with a potent and stable GLP-2 compound is able to inhibit key parameters of the inflammatory manifestations of mesenteric I/R. The protection by a GLP-2 compound is abolished by co-administration with the protein synthesis inhibitor, cycloheximide. This shows that the effect of the GLP-2 compound involves de novo protein synthesis.

1-10. (canceled)

1. A method for the treatment of ischemia-reperfusion injury in a subject comprising administering to said subject a composition comprising a therapeutically effective amount of a GLP-2 compound, whereby at least one symptom of ischemia-reperfusion injury is alleviated.

12. (canceled)

13. The method of claim 11, wherein the GLP-2 compound is a GLP-2 peptide comprising an amino acid sequence having at least about 75% identity to SEQ ID NO:1.

14. The method of claim 13, wherein the subject is a human.

15. The method of claim 14, wherein the GLP-2 peptide is a GLP-2 derivative.

16. The method of claim 14, wherein the GLP-2 compound is administered substantially concurrently with the onset of ischemia.

17. The method of claim 14, wherein the patient is suffering from an ischemia-reperfusion injury in the intestine.

18. The method of claim 14, wherein the patient is suffering from an ischemia-reperfusion injury in the brain.

19. The method of claim 14, wherein the patient is suffering from an ischemia-reperfusion injury in the heart.

20. The method of claim 14, wherein the patient is suffering from an ischemia-reperfusion injury in the liver, kidney, or lung.

21. The method of claim 14, wherein the patient is suffering from an ischemia-reperfusion injury in the cornea.

22. The method of claim 14, wherein the ischemic reperfusion injury was caused by or is expected to be caused by a major organ transplant.

23. The method of claim 14, wherein the ischemic reperfusion injury is caused by angina, coronary artery bypass, aneurysm, or myocardial infarction.

24. The method of claim 14, wherein the ischemic reperfusion injury was caused by trauma.

25. The method of claim 14, wherein the ischemic reperfusion injury was caused by surgery.

26. The method of claim 14, wherein the GLP-2 compound is a GLP-2 peptide comprising an amino acid sequence having at least about 85% identity to SEQ ID NO:1.

27. A method for preventing ischemia-reperfusion injury in a subject (a) about to undergo a procedure capable of causing ischemia-reperfusion injury or (b) that has been subject to a procedure in which ischemia-reperfusion injury can occur but has not yet occurred comprising administering to said subject an amount of a GLP-2 compound effective to prevent at least one symptom of ischemia-reperfusion injury.

28. The method of claim 27, wherein the GLP-2 compound is a GLP-2 peptide comprising an amino acid sequence having at least about 75% identity to SEQ ID NO:1.

29. The method of claim 28, wherein the subject is a human.

30. The method of claim 29, wherein the subject is about to undergo or has undergone a major organ transplant.

31. The method of claim 28, wherein the GLP-2 compound is a GLP-2 peptide comprising an amino acid sequence having at least about 85% identity to SEQ ID NO:1.
